(19) United States
(12) Patent Application Publication
(10) Pub. No.: US 2026/0215437 A1
(43) Pub. Date: Jul. 30, 2026

(54) COMPOSITIONS AND METHODS FOR PROVIDING PLANTS WITH TOLERANCE TO DROUGHT CONDITIONS

(71) Applicant: KING ABDULLAH UNIVERSITY OF SCIENCE AND TECHNOLOGY, Thuwal (SA)

(72) Inventors: Heribert HIRT, Thuwal (SA); Maged SAAD, Thuwal (SA); Khairiah Mubarak ALWUTAYD, Thuwal (SA)

(21) Appl. No.: 19/148,690

(22) PCT Filed: Jan. 18, 2024
(86) PCT No.: PCT/IB2024/050496

§ 371 Date: Jul. 17, 2025

Related U.S. Application Data
(60) Provisional application No. 63/480,363, filed on Jan. 18, 2023.

(51) Int. Cl. A01N 63/27; A01N 25/24; A01P 21/00

(52) U.S. Cl. CPC A01N 63/27; A01N 25/24; A01P 21/00

(57) ABSTRACT

Compositions of Pseudomonas argentinensis strain SA190 (hereinafter, SA190), alone or in combination with Enterobacter sp. SA187 are provided. The compositions can be used to provide seeds or plants with resistance or tolerance to drought stress. Compositions containing SA190, alone or in combination with SA187 can be used to enhance plant development and yield under environmental stress conditions resulting from drought. Plant substrates for example, soil, peat, compost, vermiculite, perlite, sand, clay and combinations thereof containing effective amounts of SA190 and/or SA187 to confer drought tolerance to the plant following colonization of the plant are also provided.

### FIELD OF THE INVENTION

The invention is generally directed to bacterial compositions and methods of use to drought tolerance or resistance in plants.

### BACKGROUND OF THE INVENTION

Drought is one of the greatest worldwide environmental constraints in agriculture. The percentage of the planet affected by drought has more than doubled in the last 40 years and in the same timespan droughts have affected more people worldwide than any other natural hazard. Climate change increases the frequency, severity, and duration of drought in many parts of the world, making crop resistance to drought stress a major goal of agricultural biotechnology. Drought stress adversely affects the plant growth which results in reduced crop yield and ultimately to critical food shortages or famines (Seleiman et al, 2021). Improving crop resistance to drought stress is a long-standing goal of agricultural biotechnology. Although progress has been made to understand and identify important molecular players of drought stress in model plants, success in translating knowledge from model plants to crops has so far been limited (Cominelli et al., 2013). This is partially due to the fact that drought stress is a complex trait causing dramatic changes in many physiological plant parameters (Zhu, 2002). After all, the different stress conditions that coexist in open field agriculture go beyond simple arithmetic of the tolerance to single stresses (Suzuki et al, 2014).

Drought stress drastically changes plant morphology, biochemistry and physiology leading to sever impact on plant growth and yield. The first physiological response of plants to drought is the closure of stomata to avoid water loss via transpiration (Pirasteh-Anosheh et al, 2016). The decrease in transpiration rate leads to a decrease in relative water content (RWC) of plants (Giday et al, 2014). Stomatal closure also decreases plant photosynthetic efficiency by decreasing intracellular CO2 concentrations (Lisar et al, 2016).

Moreover, drought stress tolerance is dependent on the specific interactions between genotype and environment and is controlled by many genes, most of which make small contributions to drought tolerance. Finally, the different stress conditions that coexist in open field agriculture go beyond simple arithmetic of single stress tolerance traits (Suzuki et al., 2014).

Arid regions cover about one quarter of the Earth's land surface and encompass many of the challenges for increasing agricultural productivity. In contrast to better known dryland farming, desert agriculture can function only when crop plants are under irrigation—usually with underground water (Cresewell, 1998). Although deserts appear to be hardly inhabitable, a wide diversity of organisms, including a range of plant species, has adapted to these extreme water-limiting conditions, providing an ideal reservoir to isolate beneficial bacteria enhancing plant tolerance towards drought stress (de Zelicourt et al., 2013).

### BRIEF SUMMARY OF THE INVENTION

It has been discovered that Pseudomonas argentinensis strain SA190 (hereinafter, SA190), alone or in combination with Enterobacter sp. SA187 (hereafter named SA187) can provide seeds or plants with resistance or tolerance to drought stress. Compositions containing SA190, alone or in combination with SA187 can be used to enhance plant development and yield under environmental stress conditions resulting from drought.

One embodiment provides an isolated culture of SA190. This embodiment includes compositions containing SA190, which can be in liquid or solid form, such as powder forms.

In another embodiment, the SA190 or SA187 is genetically modified to express one or more heterologous genes or proteins. P. argentinensis strain SA190 was deposited under terms in accordance with the Budapest Treaty with the Collection Nationale de Culture de Microorganismes, Institut Pasteur, having an address of 25 rue du Docteur Roux, F-75724 Paris Cedex 15, on Nov. 24, 2021, with the following deposit number: CNCM 1-5780: P. argentinensis. nov SA190 strain. was redeposited under terms in accordance with the Budapest Treaty with the Collection Nationale de Culture de Microorganismes, Institut Pasteur, having an address of 25, Rue du Docteur Roux, F-75724 Paris Cedex 15 on Aug. 24, 2022 with the following deposit number: CNCM 1-5880.

Enterobacter sp. SA187 strain was deposited under terms in accordance with the Budapest Treaty with the American Type Culture Collection (ATCC), having an address of P.O. Box 1549, Manassas, VA 20110 USA, on Sep. 24, 2019, with the following deposit number: PTA-126210.

Another embodiment provides a plant substrate containing an effective amount of SA190 and/or SA187 to confer drought tolerance to the plant following colonization of the plant. In a preferred embodiment suitable plant substrates include, but are not limited to soil, peat, compost, vermiculite, perlite, sand, clay, and combinations thereof.

Another embodiment provides a plant seed coated with an effective amount of SA190 alone or in combination with SA187 to confer drought tolerance to the plant following germination of the seed. In a preferred embodiment suitable plant substrates include, but are not limited to soil, peat, compost, vermiculite, perlite, sand, clay and combinations thereof. The coating can contain one or more additional plant growth-promoting bacteria or rhizobacteria.

Still another embodiment provides a plant inoculated with an effective amount of SA190, alone or in combination with SA187 to confer drought tolerance to the plant, and optionally including one more additional plant growth-promoting bacteria or rhizobacteria.

Another embodiment provides a plant root coated with P. argentinensis SA190, alone or in combination with SA187 and optionally including one more additional plant growth-promoting bacteria or rhizobacteria.

Still another embodiment provides a method of providing a seed or plant with resistance to drought conditions by coating the seed or a root of the plant with an effective amount of SA190, alone or in combination with SA187 to provide the seed or plant with resistance to the drought conditions.

Yet another embodiment provides a method of improving growth of a seed or plant under drought conditions by growing the seed or plant in a plant substrate, wherein the plant substrate comprises an effective amount of SA190, alone or in combination with SA187 to colonize the seed or a root of the plant to provide resistance to conditions of drought, to the seed or plant.

Another embodiment provides a method of improving tolerance of a seed or plant to drought conditions by coating the seed or root of the plant with an effective amount of SA190, alone or in combination with SA187 to provide the plant with resistance to drought conditions.

Still another embodiment provides a method of improving tolerance of a seed or plant against drought conditions by growing the seed or plant in a plant substrate comprising an effective amount of SA190, alone or in combination with SA187. to provide the seed or plant with tolerance to the drought conditions.

Yet another embodiment provides a method of providing a plant with tolerance to drought conditions by inoculating the plant's rhizosphere SA190, alone or in combination with SA187.

Another embodiment provides a seed coating composition containing SA190, alone or in combination with SA187. The SA190, alone or in combination with SA187 can be encapsulated with a non-toxic, biodegradable coating. The seed coating composition can also contain a coating adhesive. Exemplary seed coating compositions contain gelatin, cellulose, alginate, xanthum, or a combination thereof. Certain seed coating compositions can have multiple layers.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A-1I show that Pseudomonas argentinensis strain SA190 enhances drought tolerance in Arabidopsis and crops. FIG. 1A. Graphical representation of work flow for plant screening assay under 25% PEG. FIG. 1B. Growth of non-colonized and SA190-colonized Arabidopsis plants grown under drought stress proxy for 21 days (½ MS+25% PEG) or ambient conditions for 14 days (½ MS). FIG. 1C. Total fresh weight, FIG. 1D. Total dry weight, FIG. 1E. Primary root length and FIG. 1F. Lateral roots density of 21-day-old, non-colonized (Mock) and SA190-colonized plants upon growth for 16 days on ½ MS or ½ MS+25% PEG. FIG. 1G. Survival percentage of mock- and SA190-colonized plants in jiffy pots grown upon watering for 2 weeks, no watering for 3 weeks and after one week of re-watering. All plots represent the mean of 3 biological replicates (n=36). Error bars represent SE. Asterisks indicate the statistical differences based on the Student's t-test (*p<0.05; **p<0.01; ***p<0.001). FIG. 1H. Alfalfa biomass yield in a growth chamber with normal or drought (10 days without irrigation). The differences in yield between different treatments are indicated in percent (%) and the statistical significance by t-test at p<0.05 by asterisks (*). Non-colonized plants are indicated in grey and SA190-colonized plants in green.

FIGS. 2A-2F. Transcriptome analysis and role of ABA signaling in SA190-induced PEG drought proxy tolerance. FIG. 2A. SA190 colonization levels in Arabidopsis under normal (½ MS) and drought proxy (½ MS+25% PEG) conditions as quantified by colony forming units of bacteria per mg of plant fresh weight. FIGS. 2B and 2C. GO term analysis of differentially expressed genes of SA190-colonized (PEG+190) compared with non-colonized (PEG) plants under drought proxy conditions (½ MS+25% PEG). FIGS. 2D and 2E. Total fresh weight and root fresh weight of 21 days old non-colonized (Mock) and SA190-colonized (SA190) Col-0, aba2-1 and qpyr/pyl plants grown under drought proxy conditions (½ MS+25% PEG). FIGS. 2F and 2G. Primary root length of 21-day-old, mock- and SA190-colonized Col-0, aba2-1 and qpyr/pyl plants upon growth for 16 days on ½ MS+25% PEG.

FIG. 3A. Lateral root density of 21-day-old, mock- and SA190-colonized Col-0, aba2-1 and qpyr/pyl plants upon growth for 16 days on ½ MS+25% PEG FIG. 3B-C. SA190-induced aquaporin gene expression is ABA-dependent. FIG. 3B. Relative gene expression of aquaporin PIP and TIP genes by qRT-PCR analysis normalized to tubulin levels in 21-days old mock- and SA190-colonized roots of Arabidopsis grown for 16 days on ½ MS with or without 25% PEG. FIG. 3C. Relative gene expression of aquaporin PIP and TIP genes by qRT-PCR analysis in Col-0, aba2-1 and qpyr/pyl mutants normalized to tubulin levels in 21-days old mock- and SA190-colonized roots of Arabidopsis grown for 16 days on ½ MS with or without 25% PEG. Values represent the means of three biological experiments. Error bars indicate SE. All plots represent the mean of 3 biological replicates. Asterisks indicate a statistical difference based on multiple unpaired t-test (*p<0.05; **p<0.01; ***p<0.001).

FIG. 4. SA190 primes aquaporin genes via H3K4me3 enrichment. Relative enrichment of the H3K4me3 mark at the indicated gene loci of selected PIPs and TIPs of non-colonized (Mock) and SA190-colonized (SA190) plants in Col-0 and aba2-1 mutant as determined by chromatin immunoprecipitation-qPCR (ChIP-qPCR). The regions targeted for amplification are labelled as R1, R2 and R3. R1 encompasses 5′-UTR, R2 is close to the transcription start site (TSS) while R3 is outside the gene body. Amplification values were normalized to input and H3 and region 3 (R3) of non-colonized (Mock) Col-0 plants. The plots represent values from 2 biological replicates.

FIG. 5A-5J. Role of SA190 colonization in aquaporin and plant PEG drought stress proxy physiology. FIG. 5A-5B. Total fresh weight of 21 days old non-colonized-(Mock) and SA190-colonized (SA190) Col-0, tip2;1 and qpip plants grown for 16 days under normal (½ MS) or drought stress proxy conditions (½ MS+25% PEG). FIG. 5C. Quantification of SA190 colonization efficiency in 21 days old-SA190-colonized (SA190) Col-0, tip2;1 and qpip plants grown for 16 days under normal (½ MS) or drought conditions (½ MS+25% PEG). FIG. 5D-5E. Total fresh weight of mock- and SA190-colonized 21-day-old Arabidopsis plants grown for 16 days under normal on (½ MS) or PEG drought stress proxy (½ MS+25% PEG) conditions supplemented with the aquaporin inhibitors HgCl₂ (38.2 μM) or AgNO₃ (5 μM). FIG. 5F. The percentage of leaf relative water content (RWC) of 21-days old mock- or SA190-colonized plants grown for 16 days under drought stress proxy (½ MS+25% PEG) conditions. The RWC was calculated as the formula RWC (%)=[(FW−DW)/(TW−DW)]×100, where FW is fresh weight, DW is dry weight and TW is the weight of fully turgid leaves. FIG. 5G. Transpiration rate of mock- or SA190-colonized 21 days old plants after transfer of 5-day-old seedlings from ½ MS to ½ MS+/−25% PEG. Transpiration was quantified as rate of water loss by calculating the changes in fresh weight for 30 min with reading at every 5 min interval. FIG. 5H-5I. Comparison of stomatal aperture of SA190- and non-colonized 21-days old plants grown for 16 days under non-stress (½ MS) or drought stress proxy (½ MS+25% PEG) conditions. Scale bar=20 μm. FIG. 5J. Water use efficiency (WUE) of 4-weeks old mock- or SA190-colonized plants under PEG drought stress proxy conditions. WUE was calculated as mg of dry weight produced per ml of water used. All plots represent the mean of 3 biological replicates. Error bars represent SE. Asterisks indicate a statistical difference based on the Student's t-test (*p<0.05; **p<0.01; ***p<0.001).

FIG. 6A-6D. SA190 enhances Arabidopsis PEG drought stress proxy tolerance. FIG. 6A. Shoot fresh weight and FIG. 6B. Root fresh weight (b) of 16-day-old mock- and SA190-colonized plants grown on ½ MS medium and ½ MS+PEG medium. FIG. 6C. Shoot dry weight and FIG. 6D. Root dry weight of 16-day-old mock- and SA190-colonized plants grown on ½ MS medium and ½ MS+PEG medium. All plots represent the mean of 3 biological replicates (n>39). Error bars represent SE. Asterisks indicate the statistical differences based on the Student's t-test (*P<0.05; **P<0.01; ***P<0.001

FIG. 7. ABA does not influence SA190 colonization of Arabidopsis. Efficiency of root colonization by SA190 in 21 days old Col-0 and aba2-1 plants grown for 16 days under normal (½ MS) or drought proxy conditions (½ MS+25% PEG).

FIGS. 8A-8D. ABA does not influence SA190-induced Arabidopsis growth and development under ambient conditions. FIGS. 8A and 8B. Total fresh weight and root fresh weight of 21 days old non-colonized (Mock) and SA190-colonized (SA190) Col-0, aba2-1 and qpyr/pyl plants grown under non stress conditions (½ MS). FIGS. 8C and 8D. Primary root length and Lateral root density of 21-day-old, mock- and SA190-colonized Col-0, aba2-1 and qpyr/pyl plants upon growth for 16 days on ½ MS plates.

FIGS. 9A and 9B. Arabidopsis-SA190 interaction is independent of the ethylene pathway. Total fresh weight of mock- and SA190-colonized 16-day-old ethylene mutants on (FIG. 9A) ½ MS or (FIG. 9B) 25% PEG. Error bars represent SE and asterisks indicate a statistical difference based on the Student's t-test (*p<0.05; **p<0.01; ***p<0.001).

FIGS. 10A-10B. Role of SA190 colonization in plant PEG drought stress proxy physiology. FIG. 10A. The percentage of leaf relative water content (RWC) of 21-days old mock- or SA190-colonized plants grown for 16 days under normal (½ MS) conditions. The RWC was calculated as the formula RWC (%)=[(FW−DW)/(TW−DW)]×100, where FW is fresh weight, DW is dry weight and TW is the weight of fully turgid leaves. FIG. 10B. Comparison of stomatal aperture of SA190- and non-colonized 21-days old plants grown for 16 days under non-stress (½ MS) conditions. Scale bar=20 μm.

FIG. 11. Water use efficiency (WUE) of 4-weeks old mock- or SA190-colonized plants grown under non-stress (½ MS) conditions. WUE was calculated as mg of dry weight produced per ml of water used. All plots represent the mean of 3 biological replicates. Error bars represent SE. Asterisks indicate a statistical difference based on the Student's t-test (*p<0.05; **p<0.01; ***p<0.001).

FIGS. 12-13 shows the results of alfalfa (FIG. 12) and cucumber (FIG. 13) crops subjected to 25% and 30% less water irrigation, respectively. Alfalfa was grown on sandy soil open field agriculture, while cucumber was grown in sand soil-less greenhouse culture conditions. The differences in yield between different treatments are indicated in percent (%) and significance at P<0.05 is indicated by asterisks (*).

FIGS. 14A-14D show the effect of SA190, SA187 or the combination of SA190 and SA187 inoculation on plant total FW (FIG. 14A), shoot FW (FIG. 14B) under drought stress (under drought stress (½ MS+25% PEG).

FIGS. 15A and 15B show quantification of the levels of phytohormones ABA (FIG. 15A) and OPDA (FIG. 15B) in shoot under normal and drought stress conditions. The levels indicate the μg amounts of hormones present per 1 g of fresh weight. FIG. 15C shows detection of hydrogen peroxide accumulation by DAB staining in 16-day-old non- or SA190-plants colonized under normal and drought stress. FIGS. 15D and 15E show qPCR expression analysis of ROS marker genes RBOHD (E) and RBOHF (F) normalized to Tubulin levels in 16-days-old mock- and SA190-colonized Arabidopsis seedling grown on ½ MS with or without 25% PEG. Values represent means of three biological experiments. Error bars indicate SE. FIGS. 15F and 15G show quantification of the levels of phytohormones ABA (FIG. 15A) and OPDA (FIG. 15B) in roots under normal and drought stress conditions. The levels indicate the μg amounts of hormones present per 1 g of fresh weight.

### DETAILED DESCRIPTION OF THE INVENTION

The disclosed methods and compositions are based on a discovery that Pseudomonas argentenensis strain SA190, an endophytic bacterium isolated from root nodules of the indigenous desert plant Indigofera argentea massively increases drought tolerance in plants. SA190 could enhance drought tolerance of Arabidopsis. Physiological and genetic analysis showed that SA190 maintains growth and photosynthesis by enhancing plant water use efficiency. Although drought-induced ABA levels were strongly reduced by SA190 colonization, mutants in ABA biosynthesis or signaling were compromised in SA190-induced drought tolerance. Transcriptome analyses uncovered that SA190-induces the expression of multiple aquaporin genes in roots and shoots of Arabidopsis and that inhibition of aquaporin functioning abrogates SA190-induced plant drought tolerance. The agronomic potential of SA190 was tested in field trials with alfalfa (Medicago sativa) under desert agriculture and cucumber in green house production conditions with both normal and reduced irrigation. Even under limited irrigation, SA190 surpassed productivity of alfalfa biomass and cucumber fruit yield, demonstrating that SA190 can help to reduce water input and hence reduce water consumption in various agricultural systems.

The disclosed methods and compositions are also based on the discovery that Enterobacter sp. SA187, improves the drought tolerance in plants conferred by SA190, or vice versa.

Thus, compositions and methods for increasing drought tolerance in plants based on these discoveries, are provided. The compositions include, but are not limited to liquid compositions containing SA190, alone or in combination with SA187, for example, whole broth culture, freeze dried compositions of SA190, alone or in combination with SA187, seeds inoculated with SA190, alone or in combination with SA187, seedlings and plant inoculated with SA190, alone or in combination with SA187, or plant substrates such as soil, peat, compost, vermiculite, perlite, sand, clay and combinations thereof, containing with SA190, alone or in combination with SA187

The disclosed method and compositions can be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### I. Definitions

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “plant substrate” refers to a substrate commonly used for growing plants, including plant seeds, plant roots and plant seedlings. Non-limiting examples of such plant substrates include, but are not limited to soil, peat, compost, vermiculite, perlite, sand, clay, and combinations thereof.

The term “culture” describes a population of microorganisms cultivated or grown in a nutrient medium.

Also as used herein the phrases “fresh nutrient medium”, “suitable nutrient medium” and the like mean an aqueous solution or suspension of nutrients which are necessary and sufficient to support the growth of the disclosed microorganisms.

By the term “effective amount” of a composition as provided herein is meant a nontoxic but sufficient amount of the composition to provide the desired result. As will be pointed out below, the exact amount required will vary from plant to plant, depending on the species and the severity of the disease that is being treated, the mode of administration, and the like. Thus, it is not possible to specify an exact “effective amount.” However, an appropriate effective amount can be determined by one of ordinary skill in the art using only routine experimentation.

“Plant growth-promoting rhizobacteria (PGPR)” refers soil bacteria that colonize the roots of plants following inoculation onto seed and that enhance plant growth (Kloepper, Joseph W.; Schroth, Milton N., Proceedings of the 4th International Conference on Plant Pathogenic Bacteria (Angers, France: Station de Pathologie Végétale et Phytobactériologie, INRA) 2:879-882 (1978); Aziz, et al., Malaysian Journal of Microbiology 8 (1): 47-50 (2012)).

The term “plant substrate” refers to a substrate commonly used for growing plants, including plant seeds, plant roots and plant seedlings. Non-limiting examples of such plant substrates include, but are not limited to soil, peat, compost, vermiculite, perlite, sand, clay, and combinations thereof.

As defined herein, “whole broth culture” refers to a liquid culture containing both cells and media. If bacteria are grown on a plate the cells can be harvested in water or other liquid.

The term “plant material” refers to leaves, stems, roots, flowers or flower parts, fruits, pollen, egg cells, zygotes, seeds, cuttings, cell or tissue cultures, or any other part or product of a plant.

The term “plant cell culture” refers to cultures of plant units such as, for example, protoplasts, cell culture cells, cells in plant tissues, pollen, pollen tubes, ovules, embryo sacs, zygotes and embryos at various stages of development.

The term “plant” is used in it broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

“Seed germination” refers to growth of an embryonic plant contained within a seed resulting in the formation and emergence of a seedling.

### I. Compositions

Compositions containing SA190 alone or in combination with SA187 are provided that can be used to provide seeds and plants with resistance or tolerance to drought conditions.

### A. SA190

i. Liquid Compositions

A substantially pure culture or whole cell broth containing Pseudomonas argentenensis strain SA190 is provided. The compositions include SA190 in combination with a suitable bacterial cell culture medium such as LB broth (Lennox L Broth Base), MOPS (3-(N-Morpholino) Propane-Sulfonic Acid) medium, M9 minimal medium commercially available from ThermoFisher Scientific, for example, or M63 minimal medium. SA190 can grow in King agar KB (King's B) media, TSA media as well as SA190 can grow in Trypticase soy agar or tryptone soya agar (TSA) and Trypticase soy broth or tryptone soya broth (TSB). The composition in one preferred embodiment is a plant tissue culture medium, preferably containing agar. Agar, a polysaccharide obtained from seaweeds, is of universal use as a gelling agent for preparing semi-solid and solid plant tissue culture media. Plant tissue and cell culture refers to the sterile growth and multiplication of plant cells, tissues, and organs in vitro. Plant cells cultured with nutrient media in an artificial environment can be clonally propagated at scale, to more quickly produce mature plants. In many common plant cell culture processes such as seed culture, meristem culture, callus culture, bud culture, and another culture, tissues are placed on a gel substrate such as Murashige and Skoog (MS media, MSO, or MS0), Whites basal medium, or Gamborg B5 medium. Gamborg's B5 medium is an optimized culture medium for in vitro plant cell, tissue, and organ culture. It contains a mixture of inorganic salts, vitamins, and carbohydrates. We offer Gamborg's B5 medium with minimal organics, Gamborg's vitamin mix, and Gamborg's B5 basal salt mix. Tissues may also be placed into a liquid medium, as is the case with cell suspension culture. The plant culture media formulation may include macronutrients, micronutrients, vitamins and organic supplements, amino acids and nitrogen supplements, plant growth hormones and plant growth regulators (PGRs), and will vary depending on specific plant needs.

ii. Freeze Dried

Freeze dried micro-organism compositions containing SA190 alone or in combination with SA187 are disclosed. The freeze dried micro-organism composition further comprises additional components such as at least a protectant. Cells which are freeze-dried in the presence of protective agents are better suited to maintain their viability and stability than cells which are freeze-dried without the addition of said protective agents. So generally a protectant is mixed with fresh cell concentrate prior the freeze-drying step.

The protectant (or protective agent) may be selected from cryoprotectants, lyoprotectants and mixtures thereof.

Cryoprotectants can be defined as substances used to prevent or reduce damage to cells or tissues during freezing and to further prevent or reduce damage during frozen storage. Cryoprotectants often work by changing the characteristics of ice.

Lyoprotectants can be defined as substances used to prevent or reduce damage to cells or tissues during desiccation or freeze drying and optionally to further prevent or reduce damage during dry storage. Lyoprotectants often work by protecting biological structures after water has been removed.

Some substances may act as both cryoprotectants and lyoprotectants. Some substances may act as both cryoprotectants and lyoprotectants.

Various protective agents have thus been used in the art, with varying degrees of success. These protective agents include antioxidants, amino acids, proteins, protein hydrolysates, certain polymers, skim milk, glycerol, carbohydrate such as oligosaccharides and polysaccharides. Suitable examples of proteins or protein hydrolysates include the ones selected from the group consisting of Malt extract, Skimmed milk powder, Whey powder, Yeast extract, Gluten, Collagen, Gelatin, Elastin, Keratin, and Albumins.

The protectant may also preferably be a compound involved in bio-synthesis of nucleic acids.

The protectant can be a carbohydrate. Preferred suitable carbohydrates include the ones selected from the group consisting of pentoses (e.g. ribose, xylose), hexoses (e.g. fructose, mannose, sorbose), disaccharides (e.g. sucrose, trehalose, melibiose, lactulose), oligosaccharides (e.g. raffinose), oligofructoses (e.g. Actilight, fribroloses), polysaccharides (e.g. maltodextrins, Xanthan Gum, pectin, alginate, Microcrystalline cellulose, Dextran, PEG), and sugar alcohols (sorbitol, manitol, lactitol, maltitol, glycol, glycerol, erythritol, threitol, arabitol, xylitol, ribitol, dulcitol, iditol, isomalt and polyglycitol). In one aspect, the carbohydrate is a carbohydrate with a molecular weight (MW) from 150 to 100000 g/mol, more preferably 250 to 100000 g/mol, even more preferably from 300 to 40000 g/mol and most preferably from 500 to 15000 g/mol. Preferably, the protectant is selected from trehalose sucrose and maltodextrin. In one aspect of the invention the protectant is trehalose.

The protectant may be added to the composition of the present invention in any suitable amount to provide the desired protection against, for example freezing or freeze drying. Typical amounts of protectant are known to one skilled in the art and may be readily identified by one skilled in the art. In one aspect protectant is provided in the present freeze dried micro-organism composition in an amount of less than 90 wt % based on the total weight of the freeze dried micro-organism composition, such as in an amount of less than 85 wt % based on the total weight of the freeze dried micro-organism composition, such as in an amount of less than 80 wt % based on the total weight of the freeze dried micro-organism composition, such as in an amount of less than 75 wt % based on the total weight of the freeze dried micro-organism composition, such as in an amount of less than 70 wt % based on the total weight of the freeze dried micro-organism composition, such as in an amount of less than 65 wt % based on the total weight of the freeze dried micro-organism composition, such as in an amount of from 40 to 80 wt % based on the total weight of the freeze dried micro-organism composition, such as in an amount of from 50 to 70 wt % based on the total weight of the freeze dried micro-organism composition, such as in an amount of at least 10 wt % based on the total weight of the freeze dried micro-organism composition, such as in an amount of at least 20 wt % based on the total weight of the freeze dried micro-organism composition, such as in an amount of at least 30 wt % based on the total weight of the freeze dried micro-organism composition, such as in an amount of at least 40 wt % based on the total weight of the freeze dried micro-organism composition, such as in an amount of at least 50 wt % based on the total weight of the freeze dried micro-organism composition.

The protectant may be added to the micro-organism composition such that it is incorporated in the freeze dried micro-organism composition. In a preferred aspect the protectant is added to the micro-organism composition prior to freezing.

In another embodiment, the process comprises a further step of addition of a protectant before step (i) and/or before the step of freezing (i′).

Freeze-drying or lyophilization is generally recognized as an effective procedure for preserving microorganisms. In this process, microorganisms are dried rapidly while they are frozen. Freeze-drying can be performed using different techniques. In particular freeze-drying can be performed by tray drying. In this process, the stabilized cell concentrate is loaded directly into freeze dryer trays. The cells are frozen by contact with shelves maintained at a freezing temperature and freeze-dried in a commercial freeze-drier. The resulting cake may then be milled to make a powder, for example which is used in probiotic blends. Another common freeze-drying process used to preserve cultures is to freeze-dry them in frozen pellet form. The frozen pellets may be formed by dripping stabilized culture onto a chilled surface (such as a chilled barrel or a chilled belt) or into liquid nitrogen. The frozen pellets can be produced and stored independently of freeze-drier availability, and can be easily loaded into freeze drier trays. The resulting dry pellets may then be milled to make a powder, for example which is used in probiotic blends.

Methods for preparing freeze dried microorganism composition are known in the art. See for Example, EP 0145197, which discloses method of freeze-drying microorganisms and methods of culturing freeze-dried microorganisms. See also, U.S. Pat. No. 9,554,583

### B. Seeds, Seedlings and Plants

Preferred seeds and plants that can be inoculated with or coated with SA190 alone or in combination with SA187 include, but are not limited to wheat, maize, soybean, oat, barley, potato, and sugar beets.

### Co-Inoculation

Rhizosphere microorganisms, particularly beneficial bacteria and fungi, can improve plant performance under stress environments and, consequently, enhance yield both directly and indirectly (C. Dimkpa, et al, Plant, Cell and Environment, 32 1682-1694 (2009)). Some plant growth-promoting rhizobacteria (PGPR) may exert a direct stimulation on plant growth and development by providing plants with fixed nitrogen, phytohormones, iron that has been sequestered by bacterial siderophores, and soluble phosphate (R. Hayat, et al., Annals of Microbiology, 60 579-598 (2010)). Others do this indirectly by protecting the plant against soil-borne diseases, most of which are caused by pathogenic fungi (B. Lutgtenberg and F. Kamilova, Annual Review of Microbiology, 63 541 556 (2009)).

Bacteria of diverse genera such as Arthrobacter, Azotobacter, Azospirillum, Bacillus, Enterobacter, Pseudomonas and Serratia (E. J. Gray and D. L. Smith, Soil Biology & Biochemistry, 37 395 412 (2005)), as well as Streptomyces spp. (Dimkpa, C. et al., Canadian Journal of Microbiology, 54:163 172 (2008)) have been identified as PGPR and can be combined with Pseudomonas argentinensis strain SA190 in the disclosed compositions.

Plants and seeds can be co-inoculated with Pseudomonas argentinensis strain SA190 alone or in combination with SA187, and one or more other plant growth-promoting bacteria or rhizobacteria to provide the seeds or plants with resistance or tolerance to drought conditions. Co-inoculation is based on mixed inoculants, combination of microorganisms that interact synergistically, or when microorganisms such as Azospirillum are functioning as “helper” bacteria to enhance the performance of other beneficial microorganisms. In the rhizosphere the synergism between various bacterial genera such as Bacillus, Pseudomonas and Rhizobium has been demonstrated to promote plant growth and development. Compared to single inoculation, co-inoculation can improve the absorption of nitrogen, phosphorus and mineral nutrients by plants.

Suitable bacteria that can be co-inoculated with SA190 and SA187 include but are not limited to Pseudomonas putida, Pseudomonas aeruginosa, Klebsiella sp., Enterobacter asburiae, Rhizobium sp. (pea), Mesorhizobium sp., Acinetobacter spp., Rhizobium sp. (lentil), Pseudomonas sp. A3R3, Psychrobacter sp. SRS8, Bradyrhizobium sp., Pseudomonas aeruginosa 4EA, Pseudomonas sp., Ochrobactrum cytisi, Bacillus species PSB 10, Paenibacillus polymyxa, Rhizobium phaseoli, Rahnella aquatilis, Pseudomonas fluorescens, Ralstonia metallidurans, Azospirillum amazonense, Serratia marcescens, Enterobacter sp., Burkholderia, Pseudomonas jessenii, Azotobacter sp., Mesorhizobium ciceri, Azotobacter chroococcum, Klebsiella oxytoca, Pseudomonas chlororaphis, Bacillus subtilis, Gluconacetobacter, diazotrophicus, Brevibacillus spp., Bravibacterium sp., Xanthomonas sp. RJ3, Azomonas sp. RJ4, Pseudomonas sp. RJ10, Bacillus sp. RJ31, Bradyrhizobium japonicum, Variovorax paradoxus, Rhodococcus sp., Flavobacterium, Sphingomonas sp, Mycobacterium sp, Rhodococcus sp, Cellulomonas sp., Azospirillum sp., Azospirillum brasilense, Rhizobium meliloti, Kluyvera ascorbata, Rhizobium cicero, Rhizobium leguminosarum, Paenibacillus polymyxa strain A26, a Alcaligenes faecalis strain AF, and combinations thereof.

Additional bacteria that can be inoculated with Enterobacter endophyticus sp. include those listed in Table 1.

TABLE 1
Bacterial Inoculates (From Bianco Carmen and Defez Roberto (2011).
Soil Bacteria Support and Protect Plants against Abiotic Stresses,
Abiotic Stress in Plants - Mechanisms and Adaptations, Prof. Arun
Shanker (Ed.), ISBN: 978-953-307-394-1, InTech, DOI: 10.5772/23310)

| Stress type | Bacterial inoculate | Plant Species |
|---|---|---|
| Salt | Pseudomonas | Rice (Oryza sativa) |
| | pseudoalcaligenes, | |
| | Bacillus pumilus | |
| Salt | Bacillus megaterium | Maize (Zea maize L.) |
| Salt | Azospirillum brasilense | Barley (Hordeum vulgare) |
| Salt | Pseudomonas mendocina | Lettuce (L. sativa L. cv. Tafalla) |
| Salt | Azospirillum sp. | Pea (Phaseolus vulgaris) |
| Salt | Bacillus subtilis | Arabidopsis thaliana |
| Salt | Pseudomonas syringae, | Maize (Zea maize) |
| | Pseudomonas fluorescens, | |
| | Enterobacter aerogenes | |
| Salt | P. fluorescens | Groundnut (Arachis hypogaea) |
| Salt | Azospirillum | Lettuce (Lactuca sativa) |
| Salt | Achromobacter piechaudii | Tomato (Lycopersicon esculentum) |
| Salt | Aeromonas | Wheat (Triticum aestivum) |
| | hydrophila/caviae | |
| | Bacillus insolitus, Bacillus sp. | |
| Salt | Azospirillum | Maize (Z. maize) |
| Salt | A. brasilense | Chickpeas (Cicer arietinum), |
| | | faba beans (Vicia faba L.) |
| Drought | Pseudomonas spp. | Maize (Zea mays L. cv. Kaveri) |
| Drought | Pseudomonas spp. | Asparagus (Asparagus officinalis L.) |
| Drought | Pseudomonas mendocina | Lettuce (Lactuca sativa L.) |
| Drought | Rhizobium tropici, | Common bean (Phaseolus vulgaris L.) |
| | Paenibacillus polymyxa | |
| Drought | Bacillus | Lettuce (Lactuca sativa L.) |
| Drought | Ensifer meliloti bv. | Bean (Phaseolus vulgaris cv. |
| | mediterranense | Flamingo) |
| Drought | Bradyrhizobium elkanii | Flat crown (Albizia adianthifolia) |
| Drought | Achromobacter piechaudii | Tomato (L. esculentum), |
| | | pepper (Capsicum annuum) |
| Drought | Azospirillum | Wheat (T. aestivum) |
| Drought | A. brasilense | Maize (Z. mays) |
| Drought | A. brasilense | Common bean (P. vulgaris) |
| Osmotic stress | Bacillus subtilis | Arabidopsis |
| Osmotic stress | A. brasilense | Rice (Oryza sativa L.) |
| Osmotic stress | Arthrobacter sp., Bacillus sp. | Pepper (C. annuum) |
| (45% PEG) | | |
| Osmotic stress | Azospirillum | Wheat (T. aestivum) |
| (20% PEG) | | |
| Flooding | Enterobacter cloacae, | Tomato (L. esculentum) |
| | Pseudomonas putida | |
| Temperature | Burkholderia phytofirmans | Grapevine (Vitis vinifera) |
| Temperature | Pseudomonas fluorescens, | Wheat (Triticum aestivum) |
| | Pantoea agglomerans, | |
| | Mycobacterium sp. | |
| Temperature | B. phytofirmans | Potato (Solanum tuberosum) |
| Temperature | Aeromonas hydrophila, | Soy bean (Glycine max) |
| | Serratia liquefaciens, | |
| | Serratia proteamaculans | |
| Temperature | Burkholderia phytofirmans | Grapevine (Vitis vinifera) |
| Temperature | B. phytofirmans | Potato (Solanum tuberosum) |
| Temperature | Aeromonas hydrophila, | Soy bean (Glycine max) |
| | Serratia liquefaciens, | |
| | Serratia proteamaculans | |
| Nutrient deficiency | Azospirillum sp., | Chickpea (Cicer arietinum L.) |
| | Azotobacter chroococcum, | |
| | Mesorhizobium ciceri, | |
| | Pseudomonas fluorescens | |
| Nutrient deficiency | Azotobacter coroocoocum, | Zea maize L. (Zea maize L.) |
| | Azospirillum brasilens, | |
| | Pseudomonas putida, | |
| | Bacillus lentus | |
| Nutrient deficiency | Bacillus sp., | Zea maize L. |
| | Burkholderia sp., | |
| | Streptomyces platensis | |
| Nutrient deficiency | Bacillus sp., | Zea maize L. |
| Nutrient deficiency | Bacillus polymyxa, | Zea maize L. (Zea maize cv. Felix) |
| | Mycobacterium phlei, | |
| | Pseudomonas alcaligenes | |
| Heavy metals | Sanguibacter sp., | Nicotina tabacum |
| toxicity | Pseudomonas sp. | |
| Heavy metals | Bacillus subtilis, | Oat (Avena sativa) |
| toxicity | Pantoea agglomerans | |
| Heavy metals | Pseudomonas fluorescens, | Rape (Brassica napus) |
| toxicity | Microbacterium sp. | |
| Heavy metals | Methylobacterium oryzae, | Tomato (Lycopersicon |
| toxicity | Burkholderia sp. | esculentum L.) |
| Heavy metals | Bacillus subtilis, | Rice (O. sativa) |
| toxicity | Bacillus megaterium, | |
| | Bacillus sp. | |

### C. Seed Coating Compositions

One embodiment provides a seed coating composition having at least one layer coating all or part of the seed, at least one layer contains an effective amount of Pseudomonas argentinensis strain SA190 alone or in combination with SA187 to provide the seed or the plant that grows from the seed with resistance to drought conditions. The Pseudomonas argentinensis strain SA190 alone or SA187 can be from 10⁶ Cfu/ml to 10₉ Cfu/ml of seed coating composition.

Another embodiment provides a seed coating composition containing Pseudomonas argentinensis strain SA190 alone, or in combination with SA187. The Pseudomonas argentinensis strain SA190 can be encapsulated with a non-toxic, biodegradable coating. The seed coating composition can also contain a coating adhesive. Exemplary seed coating compositions contain gelatin, cellulose, alginate, xanthum, or a combination thereof. Certain seed coating compositions can have multiple layers.

In another embodiment, the seed coating composition contains multiple layers for example, 2, 3, 4 or 5 or more layers. Pseudomonas argentinensis strain SA190 and/or one or more additional plant growth promoting bacteria can be in any or all of the layers of the seed coating composition; however, at least one of the layers of a multiple layer seed coating composition contains an effective amount of Pseudomonas argentinensis strain SA190 to provide the seed or the plant growing from the seed with resistance drought conditions. Preferably the Pseudomonas argentinensis strain SA190 or other plant growth promoting bacteria are in the layer adjacent to the surface of the seed.

In one embodiment, at least one layer contains guar gum, derivative guar, polyacrylamide, poly(methacrylic acid), poly(acrylic acid), polyacrylate, poly(ethylene glycol), phosphonate-end capped polymers, polyethyleneoxide, poly(vinyl alcohol), polyglycerol, polytetrahydrofuran, polyamide, hydroxypropyl guar, carboxymethyl guar, carboxymethyl hydroxypropyl guar, starch, derivatized (e.g., cationic) starch, corn starch, wheat starch, rice starch, potato starch, tapioca, waxy maize, sorghum, waxy sarghum, sago, dextrin, chitin, chitosan, alginate compositions, xanthan gum, carageenan gum, gum karaya, gum arabic, pectin, cellulose, hydroxycellulose, hydroxyalkyl cellulose, hydroxyethyl cellulose, carboxymethylhydroxyethyl cellulose, hydroxypropyl cellulose, a derivative of any of the foregoing or a combination of any of the foregoing. As non-limiting examples, the layer can contain a 90 wt % derivatized guar and 10 wt % starch (or derivatized starch) mixture, or a 60 wt % hydroxypropyl guar and 40 wt % carboxymethyl hydroxypropyl guar mixture.

In some embodiments, the layer can act as a carrier coating and beneficial microbes that protect the seed and emerging seedling are carried in the carrier coating. For example, alfalfa seed coating with incorporated Pseudomonas argentinensis strain SA190 is used to inoculate the field in which desired crop plants are planted or are growing.

Another embodiment provides agglomerates of seed. The agglomerate or grouping of seed is a grouping of 2 or more individual seeds together. The seeds can be for the same plant or for different plants. In another embodiment, the agglomerate is a grouping of more than 5 individual seeds together. In a further embodiment, the agglomerate is a grouping of more than 10 individual seeds together. In yet another embodiment, the agglomerate is a grouping of more than 25 individual seeds together. In yet a further embodiment, the agglomerate is a grouping of more than 50 individual seeds together. In another embodiment, the agglomerate is a grouping of more than 100 individual seeds together.

### 1. Seed Agglomerates

The agglomeration of seed can aid in the application of the seed coating composition because the seed coating composition, when using an agglomeration of seed, can be shaped or formed to be consistent in shape or form. For example, the agglomeration can be formed as spherical or substantially spherical, thus allowing the seed coating composition to be likewise substantially spherical. This can allow for improved or more consistent casting or spraying, can minimize the occurrence of blockage or clogging of the nozzles, hoses, etc. due to uneven particle size distribution. Typically, a binder or adhesive is utilized to bunch (e.g., agglomerate) the grouping of seeds together.

The agglomeration can also aid in seed or seedling establishment as a layer of the wetting agent (or other layer than affects the soil) can be concentrated to a local area of soil, thus, increasing its chance of wetting the soil surrounding the seed(s). the agglomeration can also promote survival by allowing the seeds, when germinating into seedlings, to generate sufficient force to penetrate hydrophobic areas or soil such as, for example, a hydrophobic (i.e., encrusted) soil surface

In one embodiment, the seed coating composition contains an agglomeration of seeds of from between 2 seeds to 100 seeds, typically between 2 to 50 seeds, typically between 2 to 25 seeds; and at least one layer selected from the group consisting a layer of a filler, a layer of a binding agent, a layer of a wetting agent, a layer of an anti-bacteria agent, a layer of an active ingredient and any combination thereof.

In one embodiment, the seed coating composition is of substantially uniform size of from between 10 micrometers and 4 mm in diameter. In another embodiment, the seed coating composition is of substantially uniform size of from between 25 micrometers and 2 mm in diameter. In a further, the seed coating composition is of substantially uniform size of from between 500 micrometers and 2 mm in diameter.

### 2. Binder

The seed coating composition can also contain a binder as one of the layers, the binder is sometimes referred to as an adhesive. In one embodiment, the binder can include but is not limited to molasses, granulated sugar, alginates, karaya gum, guar gum, tragacanth gum, polysaccharide gum, mucilage or any combination of the foregoing. In another embodiment, the binder is chosen from, but is not limited to, gelatin, polyvinyl acetates, polyvinyl acetate copolymers, polyvinyl alcohols, polyvinyl alcohol copolymers, celluloses (including ethylcelluloses and methylcelluloses, hydroxypropylcelluloses, hydroxymethyl celluloses, hydroxymethylpropyl-celluloses), polyvinylpyrolidones, dextrins, malto-dextrins, polysaccharides, fats, oils, proteins, gum arabics, shellacs, vinylidene chloride, vinylidene chloride copolymers, calcium lignosulfonates, acrylic copolymers, starches, polyvinylacrylates, zeins, carboxymethylcellulose, chitosan, polyethylene oxide, acrylimide polymers and copolymers, polyhydroxyethyl acrylate, methylacrylimide monomers, alginate, ethylcellulose, polychloroprene, syrups or any combination of the foregoing.

### 3. Active Ingredients

The seed coating compositions can also include one or more active ingredients in one or more of the layers of the coating. Compounds suitable as active ingredients, which in some embodiments form all or part of at least one layer of the seed coating composition, include but are not limited to herbicides, plant growth regulators, crop desiccants, fungicides, insecticides, insect repellants, and combinations thereof. Suitable pesticides include, for example, triazine herbicides; sulfonylurea herbicides; uracils; urea herbicides; acetanilide herbicides; and organophosphonate herbicides such as glyphosate salts and esters. Suitable fungicides include, for example, nitrilo oxime fungicides; imidazole fungicides; triazole fungicides; sulfenamide fungicides; dithio-carbamate fungicides; chloronated aromatic; and dichloro aniline fungicides. Suitable insecticides, include, for example, carbamate insecticides; organo thiophosphate insecticides; and perchlorinated organic insecticides such as methoxychlor. Suitable miticides include, for example, propynyl sulfite; triazapentadiene miticides; chlorinated aromatic miticides such as tetradifan; and dinitrophenol miticides such as binapacryl. Other active ingredients can include adjuvants, and surfactants, and fertilizers.

### 4. Filler

The seed coating composition can also include at least one filler as all or part of a layer. In one embodiment, the filler is selected from the group consisting of wood flours, clays, activated carbon, carbohydrates, sugars, dextrins, maltodextrins, diatomaceous earth, cereal flours, wheat flour, oat flour, barley flour, fine-grain inorganic solids, calcium carbonate, calcium bentonite, kaolin, china clay, talc, perlite, mica, vermiculite, silicas, quartz powder, montmorillonite or mixtures thereof.

### 5. Nutrients

The seed coating composition can also contain a nutrient such as a micronutrient or macronutrient in one or more layers of the seed coating composition. The nutrient can be in all or part of a layer. The nutrient can also be included with the grouping of seeds as part of the binder or adhesive. “Nutrient” as used herein can refer to an additive or substance utilized by plants, grasses, shrubs for plant, grass, and shrub growth, respectively. Macronutrients can be utilized in larger amounts by plants, grasses, etc. in proportionally larger amounts relative to micronutrients. Nutrients include but are not limited to manganese, boron, copper, iron, chlorine, molybdenum, and zinc, potassium, nitrogen, calcium, magnesium phosphorus and sulfur, among others. The seed coating compositions can include various combinations and relative amounts of individual macronutrients.

### D. Plant Substrates

One embodiment provides a plant substrate containing an effective amount of a species of the genus Pseudomonas to improve resistance to drought conditions in the plant. In a preferred embodiment, the Pseudomonas species is Pseudomonas argentinensis strain SA190. Suitable plant substrates include, but are not limited to soil, peat, compost, vermiculite, perlite, sand, clay and combinations thereof. Typically, the plant substrates contain 10⁶ to 10⁹ bacteria per/g of substrate

### II. Methods of Using

The disclosed methods and compositions are applicable to numerous plant including, for conferring/enhance tolerance to drought. Other uses are disclosed, apparent from the disclosure, and/or will be understood by those in the art.

As used herein, the terms “drought-resistance” or “drought-tolerance” refer to the ability of a plant to recover from periods of drought stress (i.e., little or no water for a period of days). Typically, the drought stress will be at least 5 days and can be as long as 18 to 20 days. Drought tolerance refers to the degree to which a plant is adapted to and or drought conditions. The plants in dry environments are subjected to random droughts, and it is generally impossible for them to escape from such adverse conditions. Drought resistance can be assayed according to any of a number of well-known techniques. For example, plants can be grown under conditions in which less than optimum water is provided to the plant. Drought resistance can be determined by any of a number of standard measures including turgor pressure, growth, yield and the like. In some embodiments, the methods described in the Example section, below can be conveniently used. “Enhanced drought tolerance” or “increased drought tolerance” refers to the ability of plants, plant seeds, plant cells or plant tissue treated with the disclosed to recover, thrive, survive and/or overcome drought conditions better than a control plants, plant seeds, plant cells or plant tissue not contacted with the disclosed compositions, maintained under and/or subjected to identical growth conditions. In various embodiments, the plants, plant seeds, plant cells or plant tissue treated with the disclosed compositions can have about 10%, about 20%, about 30%, about 40%, about 50% about 60%, about 70%, about 80%, about 90% or about 100% enhancement in or increase in tolerance to drought conditions as compared with the control plant. “Treated” herein is used to refer to inoculation, coating, irrigation, contact via cell/tissue culture media, contact using growth in a plant substrate etc. as the case may be.

Methods for providing seeds and plants with resistance or tolerance to drought conditions are provided. The methods include inoculating a seed or plant with an effective amount (10⁶-10⁸ bacteria/ml) of Pseudomonas argentinensis strain SA190 to provide the seed or plant with resistance to the drought conditions. The inoculation of the plant can be in the rhizosphere of the plant. The rhizosphere is the area around a plant root that is inhabited by a unique population of microorganisms. Alternatively, the plant root can be inoculated directly. In certain embodiments, the plant root is coated with a composition containing Pseudomonas argentinensis strain SA190. The method also includes contacting a plant cell with a composition containing an effective amount of Pseudomonas argentinensis strain SA190 alone or in combination with SA187. The cell can be contacted with the bacterial composition in vitro, for example, by growing the plant cells in plant cell culture medium. The cells that have been treated with bacterial compositions as disclosed herein may be grown into plants in accordance with conventional techniques. See, for example, McCormick et al. Plant Cell Reports 5:81-84 (1986).

A method for providing a seed or plant with resistance to drought condition include coating the seed or a root of the plant with an effective amount of Pseudomonas argentinensis strain SA190 to provide the seed or plant with resistance or drought conditions.

Yet another embodiment provides a method of improving growth of a seed or plant under drought conditions by growing the seed or plant in a plant substrate, wherein the plant substrate includes an effective amount of Pseudomonas argentinensis strain SA190 to colonize the seed or a root of the plant to provide drought resistance to the seed or plant.

Still another embodiment provides a method of improving tolerance of a seed or plant against drought conditions by growing the seed or plant in a plant substrate comprising an effective amount of Pseudomonas argentinensis strain SA190 to provide the seed or plant with tolerance to drought conditions.

Yet another embodiment provides a method of providing a plant with drought resistance by inoculating the plant's rhizosphere with Pseudomonas argentinensis strain SA190.

Seeds, seedlings or plants are inoculated with Pseudomonas argentinensis strain SA190 alone or in combination with SA187, in one embodiment by irrigating seeds, seedling or plant with irrigation water containing an effective amount of Pseudomonas argentinensis strain SA190 alone or in combination with SA187. The irrigation method is preferably drip irrigation.

Seeds, seedlings or plants are inoculated with Pseudomonas argentinensis strain SA190 alone or in combination with SA187 inoculated as disclosed herein can be grown in the fields, greenhouses or in vitro. In some embodiments, seeds are inoculated using a composition such as a slurry, containing Pseudomonas argentinensis strain SA190 alone or in combination with SA187. In brief, a slurry is prepared which preferably contains of sterilized peat, a broth culture of SA190, and sterilized sugar solution (10%) in the ratio 5:4:1 (w/v/v). Subsequently, plant seeds such as alfalfa seeds are coated with the slurry at a rate of 50 mL·kg-1. Plant seeds can also be mixed with the bacterial solution of SA190 (5.5×10⁷) with 5% sucrose for an effective amount of time, for example 3 hours. The seeds are subsequently left to dry for at room temperature. Exemplary plants include Cucumis sativus, or the plants listed in Table 1.

Equipment utilized to for coating seeds with the disclosed seed coating compositions include, but are not limited to drum coaters, rotary coaters, tumbling drums, fluidized beds and spouted beds to name a few. The seeds can be coated via a batch or continuous coating process.

The seeds can be separated prior to coating which, in one embodiment, utilizes mechanical means such as a sieve. The separated seeds can then be introduced into a coating machine having a seed reservoir. In one embodiment, the seeds in the mixing bowl are combined with one or more of the coatings described herein and adhered with a binder or adhesive.

In one embodiment of the process, one or more layers as described herein can be added to coat the seed or agglomeration. Outer layers can be introduced sequentially to the rotating drum.

In another embodiment, agglomerators or agglomerator devices may also be utilized. Coating is performed within a rotary coater by placing seeds within a rotating chamber, which pushes the seeds against the inside wall of the chamber. Centrifugal forces and mixing bars placed inside the coater allow the seed to rotate and mix with a coating layer. Binder or other coating materials can be pumped into the proximate center of the coater onto an atomizer disk that rotates along with the coating chamber. Upon hitting the atomizer disk, liquid adhesive is then directed outward in small drops onto the seed.

In one embodiment, seed coating techniques include, for example, seed in a rotating pan or drum. Seed is then misted with water or other liquid and then gradually a fine inert powder, e.g., Diatomaceous earth, is added to the coating pan. Each misted seed becomes the center of a mass of powder, layers, or coatings that gradually increases in size. The mass is then rounded and smoothed by the tumbling action in the pan, similar to pebbles on the beach. The coating layers are compacted by compression from the weight of material in the pan. Binders often are incorporated near the end of the coating process to harden the outer layer of the mass. Binders can also reduce the amount of dust produced by the finished product in handling, shipping and sowing. Screening techniques, such as frequent hand screening, are often times utilized to eliminate blanks or doubles, and to ensure uniform size. For example, tolerance for seed coating compositions described herein can be about 1/64th inch (0.4 mm), which is the US seed trade standard for sizing.

In yet another embodiment, the seed coating compositions and methods described herein comprises “in situ coating”. In situ coating means, in one embodiment, where a raw or non-coated seed is implanted in a hole, cavity or hollowed area in the ground and immediately or soon thereafter a coating composition is sprayed or applied directly into the hole, cavity or hollowed area to surround or partially surround the seed. Typically, the application of the seed as well as application of the coating composition are performed mechanically, but is understood that either or both of the referenced applications can be performed manually as well.

The coating can also be applied to a seed by spraying, dipping or brushing.

In one embodiment for in vitro culture, plant seeds are germinated or plant cells/tissue is cultured in plant growth medium containing an effective amount of Pseudomonas argentinensis strain SA190 alone or in combination with SA187. Plant growth media are known in the art. A preferred plant growth medium includes the MS medium, for example ½ MS agar medium containing an effective amount of Pseudomonas argentinensis strain SA190 alone or in combination with SA187, for example, between 10⁴ and 10¹² cfu, such as about 10⁶ cfu per petri dish, for example a cylindrical petri dish, with diameters ranging from 30 to 200 mm, and a height to diameter ratio ranging from 1:10 to 1:4.

A method for improving water use efficiency of a plant is also disclosed. The term “water-use efficiency” refers to the ability of a plant to grow with substantially no yield penalty under extended periods with significantly less than the normal amount of water (typically about half). Drought resistance can assayed according to any of a number of well-known techniques. In some embodiments, the methods described in the Example section, below can be conveniently used.

The methods include inoculating a seed or plant with an effective amount (10⁶-10⁸ bacteria/ml) of Pseudomonas argentinensis strain SA190 to provide the seed or plant with improved water use efficiency. The inoculation of the plant can be in the rhizosphere of the plant. The rhizosphere is the area around a plant root that is inhabited by a unique population of microorganisms. Alternatively, the plant root can be inoculated directly. In certain embodiments, the plant root is coated with a composition containing Pseudomonas argentinensis strain SA190. The method also includes contacting a plant cell with a composition containing an effective amount of Pseudomonas argentinensis strain SA190 alone or in combination with SA187. The cell can be in contact with the bacterial composition in vitro, for example, by growing the plant cells in plant cell culture medium. The cells that have been treated with bacterial compositions as disclosed herein may be grown into plants in accordance with conventional techniques. See, for example, McCormick et al. Plant Cell Reports 5:81-84(1986).

The method can include coating the seed or a root of the plant with an effective amount of Pseudomonas argentinensis strain SA190 to provide the seed or plant with improved water use efficiency. The method can also include growing the seed or plant in a plant substrate, wherein the plant substrate includes an effective amount of Pseudomonas argentinensis strain SA190 to colonize the seed or a root of the plant to improve water use efficiency. The method can include inoculating the plant's rhizosphere with Pseudomonas argentinensis strain SA190. Seeds, seedlings or plants are inoculated with Pseudomonas argentinensis strain SA190 alone or in combination with SA187, as disclosed above, in one embodiment by irrigating seeds, seedling or plant with irrigation water containing an effective amount of Pseudomonas argentinensis strain SA190 alone or in combination with SA187. The irrigation method is preferably drip irrigation.

In another embodiment for in vitro culture, plant seeds are germinated or plant cells/tissue is cultured in plant growth medium containing an effective amount of Pseudomonas argentinensis strain SA190 alone or in combination with SA187. Plant growth media are known in the art.

### III. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for used in a method for improving growth of a seed or plant under drought conditions, the kit comprising Pseudomonas argentinensis strain SA190. The kits also can contain SA187 and instructions for use in the disclosed methods.

The disclosed compositions and methods can be further understood through the following numbered paragraphs.

### EXAMPLES

### Material and Methods

### Plant Material, Seedling Colonization, and Stress Assay

Seeds of Arabidopsis thaliana ecotype Col-0 were surface sterilized for 10 minutes in 70% ethanol with 0.05% Triton X-100, washed three times with 100% ethanol and dried in a laminar flow hood. Sterilized seeds were scattered on half strength MS (½ MS) medium supplemented with either 100 μl of Luria Broth (LB) medium (Mock) or a fresh culture of SA190 grown in LB to an optical density of 0.21, at final concentration of 10⁸ cfu/ml. Seeds were stratified in the dark for 2 days at 4° C. and then transferred in a growth chamber set to 22° C. with a long-day photoperiod (16 h light, 8 h dark) for 5 days. The germinated seedlings (~1.0-1.5 cm root length) were then transferred to ½ MS as a normal condition or to ½ MS infiltrated with 25% Polyethylene-glycol (PEG) 8000 (Fisher Scientific, Belgium) to induce drought stress proxy. Six seedlings were used per square petri plate. The number of lateral roots (LR) was evaluated under a stereomicroscope and the root length was measured using ImageJ on the 9th day of stress treatment. Lateral root density (LRD) was calculated by dividing the number of lateral roots by the primary root length. The fresh weight (FW) of shoots and roots were taken after 16 days of stress. Dry weight (DW) was measured after drying the shoot and root tissues for 2 days at 80° C. All assays were performed in three biological replicates (bacterial colonies) and two technical replicates (petri plates). The following mutant lines were used in this study: abscisic acid mutants (aba2-1, pyr1pyl4,5,8), ethylene mutant (ein2-1, ein3-1 and acs1-1) and Aquaporin mutants tip2;1 and pip1;1 pip1;2 pip1;3 pip1,4 pip1;5 (qpip). Aquaporin inhibitors AgNO₃ (silver nitrate, Sigma) and HgCl₂ (mercury chloride, Sigma) were added to pre-cooled ½MS agar medium together with 25% PEG.

### Laser Scanning Confocal Microscopy Imaging

The SA190: GFP strain was generated as described in de Zelicourt et al., 2018. Briefly, the GFP was introduced by the E. coli SM10λpir strain carrying the GFP donor plasmid, pUX-BF13 and the pRK600 mobiliser plasmid. To determine the colonization of SA190: GFP on plant tissue the 5 day old seedlings, mounted in 100 μg/ml Propidium Iodide (PI), were used and visualized using confocal microscope ZEISS LSM880 with Airyscan with the Plan-Apochromat 10× (n.a. 0.45) objective lens. For excitation of both GFP and PI, argon-based laser with power output 6.5 or 9% for 10× or 63× objective lense, respectively were used, and samples were excited at wavelength 493-584 nm for GFP and 604-718 nm for PI. Z-stack acquisition mode with 25-70 slices (depending on the sample thickness) was used, with a maximum intensity projection afterwards using the Fiji Image J program. The ZEN blue lite edition 3.4 was used for image editing.

### Bacterial Colonization

Shoots and roots of 21-days old mock- and SA190-colonized shoots of Arabidopsis grown on ½ MS with or without 25% PEG were collected in Eppendorf tubes. Fresh weight was recorded using sensitive balance (METTLER TOLEDO). Samples were grinded using Qiagen Tissue Lyser II Sample (Disruption-11843) for 2 minutes with 500 μl of extraction buffer (10 mM MgCl₂+0.01% silwet 77), and then were incubated for 1 hour at 28° C. with shaking at 300 rpm (Eppendorf, ThermoMixer C). Samples were diluted 10-fold, and then spread on LB agar, and colony forming units (CFUs) were counted after overnight incubation at 28° C. Calculated number of CFUs was normalized to plant fresh weight.

### Stomatal Density and Aperture Measurement Assay

To measure the stomatal parameters, 5 day old seedlings inoculated with +/−SA190 on ½MS were transferred and grown further for 16 days on ½MS+/−25% PEG plates. The leaves from same developmental stage were excised from the plants and a section from middle part of the lamina excluding the mid rib was cut and mounted on a double sided tape, sticked to a microscopic glass slide, with the abaxial surface in contact with the tape. The upper green tissues were then scraped off using a scalpel and the remaining lower epidermis was imaged using an Axio Imager Z2 microscope (Zeiss) equipped with DIC optics and EC Plan-Neofluar. For measuring the stomatal aperture, the aperture width and total stomatal length of each stoma were manually measured using ImageJ and their ratios were analyzed statistically. A minimum of 30 stomata were measured from each leaf. The experiment was repeated three times, and samples were prepared from at least 3 leaves from each genotype per biological replicate.

### Transpiration Changes

To conduct the transpiration rate assays, the rosettes of 21-days old mock- and SA190-colonized shoots of Arabidopsis grown on ½ MS with or without 25% PEG were weighed at regular 5 minute intervals. The transpiration rates were calculated using the formula: WL (mg)=FWi (initial FW)—desiccated weight/min (Cohen et al, 2015).

### Leaf Relative Water Content

The 4th leaf from the rosette of six 21-days old non-colonized or SA190-colonized plants treated with 0 or 25% PEG was used to measure the relative water content (RWC). After measuring the fresh weight, leaves were placed in distilled water for 3 hours in the dark and the turgid weight was recorded. The samples were oven-dried at 70° C. for 24 hours and the LRWC was measured according to the formula:

$$LRWC ( \% ) = [ ( F W - DW ) / ( TW - D W ) ] \times 100 ( Sade et al , 2015 ) .$$

### Water Use Efficiency (WUE)

To determine the WUE for plants, sterilized 50 mL tubes were filled with a sterilize soil-perlite mixture (w:w 1:1) and 35 ml of water was added to each tube. Single 5 day old Arabidopsis seedling that had been cultured on plates with or without SA190 were transferred to the hole in the lid of individual tubes. The plants were treated as described in (Wituszynska & Karpiński, 2014). For the drought condition samples, the plants were cultivated for an extra 14 days.

### Arabidopsis Drought Assays in Soil

5-days old mock- and SA190-colonized Arabidopsis seedlings were transferred to jiffy pots in four biological replicates of 9 plants each. The plants were grown in Percival growth chambers and watered twice a week for two weeks, then watering was stopped for 3 weeks to induce drought conditions, before rewatering again for 7 days to allow the plants to recover. The plants were photographed at each step using Canon EOS 6D.

### Alfalfa Drought Assays in Soil

Control growth chamber experiments were conducted at the core lab facility at the King Abdullah University of Science and Technology (KAUST). The experiments were performed in a randomized pots (16*12 cm) arrangement of six biological replicates of 250-300 plants each. The Pots were irrigated using tap water 200 ml/week as full irrigation for 4 weeks after germination, for the drought experiments the irrigation was stopped for 10 days for the treated plants. Biomass yield was recorded after 52 days from sowing. The analysis of variance (one-way ANOVA) Including Post Hoc Tukey HSD of six replicates was performed, (P<0.05) was calculated to test the significance of differences between means. To inoculate alfalfa (Medicago sativa var. CUF 101) seeds were done according to (Daur et al, 2018) with some modification. In brief, a slurry was prepared to consist of SA190, and sterilized sugar solution (0.2%). Subsequently, alfalfa seeds were coated with the slurry at a rate of 10E8 cells per g seed. As a control, seeds were coated with a similar mixture without bacteria. The plants were sawed on stander soil and incubated in a growth chamber with temperatures 22-25° C., 16 h-8 h light-dark, photoperiod 200-300 μmol·m⁻²·s⁻¹ for 52 days until the harvest.

### RNA Extraction

Total RNA was extracted from the roots of 21-days old mock- and SA190-colonized Arabidopsis grown on ½ MS with or without 25% PEG with the Nucleospin RNA plant kit (Macherey-Nagel) following the manufacturer's recommendations. The quality and quantity of the RNA was assessed using Nanodrop-6000 spectrophotometer, 2100-Bioanalyzer (RNA integrity number greater than 8.0) and Qubit™ 2.0 Fluorometer with the RNA BR assay kit (Invitrogen).

### RNAseq Library Construction and Sequencing

In order to prepare the cDNA library, 1 μg total RNA per sample was used. The ribosomal RNA was removed using a Ribo-Zero Magnetic Kit with a 1:1 mixture of Ribo-Zero Magnetic Kit (Bacteria) and Ribo-Zero Magnetic Kit (Plant) following the manufacturer's recommendations. The quality of the library was assessed on the Agilent Bioanalyzer 2100 system. Sequencing was performed using Illumina HiSeq deep sequencing (Illumina HiSeq 2000, Illumina).

### Transcriptome Analysis

Transcriptome sequencing was performed for each library of Arabidopsis to generate 101-bp paired-end reads on Illumina HiSeq4000 Genome Analyzer platform. Low quality reads were trimmed using the Trimmomatic version 0.32 (Bolger et al, 2014) (usadellab.org/cms/?page=trimmomatic) with the following parameters: Minimum length of 36 bp; Mean Phred quality score greater than 30; Leading and trailing bases removal with base quality below 3; Sliding window of 4:15. After pre-processing the Illumina reads, the reads are mapped on to transcripts using TopHat (Trapnell et al, 2009) (ver. 2.1.1; tophat.cbcb.umd.edu/) for aligning with the genome. For TopHat, the Reference-Arabidopsis thaliana (TAIR10) genome (www.arabidopsis.org) was used as the reference sequences with maximum number of mismatches as 2. Count-based normalization implemented in DESEQ2 (Love et al, 2014) was used. To quantify the reads from the mapped alignment, featureCounts package was overlapped with each gene co-ordinates (Liao et al, 2013).

To identify the differentially expressed genes, the following parameters were used: p-value of 0.05 with a statistical correction using Benjamini Hochberg FDR of 0.05 in DESEQ2. A cut-off of 2-fold up- or down-regulation has been chosen to define differential expression. After processing the data, visualization of differential expression was done using cummerbund v2.14.0 (bioconductor.org/packages/release/bioc/html/cummerbund).

Hierarchical clustering of the quantified genes were performed using MeV 4.9.0 version (TM4, sourceforge.net/projects/mev-tm4/files/mev-tm4/MeV % 204.9.0/) utilizing the Pearson correlation method. Enriched GO term analysis was carried out using DAVID (Huang et al, 2009; Sherman et al, 2022).

qRT-PCR Analysis

Total RNA was extracted from 21-days old mock- and SA190-colonized Arabidopsis grown on ½ MS+/−25% PEG using NucleoSpin Plant RNA (Macherey Nagel) kit following the manufacturer's protocol. First strand cDNA was synthesized from 1 μg of total RNA using SuperScript III First-Strand Synthesis SuperMix kit (Invitrogen). The diluted cDNA was used to perform quantitative RT-PCR (qRT-PCR) using SsoAdvanced Universal SYBR Green Supermix (Bio-Rad). All reactions were amplified in a CFX96 Touch Real-Time PCR Detection System (BIO-RAD) at 50° C. for 2 min, 95° C. for 10 minutes, and 40 cycles of 95° C. for 10 seconds and 60° C. for 40 seconds, followed by a dissociation step to validate the PCR products. The data was analyzed using Bio-Rad CFX manager software. Tubulin was used as a housekeeping gene for normalization of gene expression levels. Analyses were performed in triplicate and were repeated three times with independent RNA samples. Primers used in this study are listed in Table 2.

TABLE 2
Primers used in this study

| | | | | |
|---|---|---|---|---|
| Gene | Gene | Primer | | |
| Code | name | code | Primer sequence F | Primer sequence R |

Primers for qPCR

| | | | | |
|---|---|---|---|---|
| AT3G61430 | PIP1-1 | AR- | TGGGATGACCACTGGGTGTTT | GGGATGGCTCTGATGACAACC |
| | | 402 | (SEQ ID NO: 1) | (SEQ ID NO: 2) |
| AT1G01620 | PIP1-3 | AR- | GGAATCTCTGGTGGGCACAT | CTCCGAGACATTGCATCACG |
| | | 404 | (SEQ ID NO: 3) | (SEQ ID NO: 4) |
| AT4G23400 | PIP1-5 | AR- | GCTGGAATCTCAGGAGGACAT | AGCTCCAAGGCACTGCATTAC |
| | | 406 | ATT (SEQ ID NO: 5) | T (SEQ ID NO: 6) |
| AT4G35100 | PIP2-7 | AR- | CGTGGGATGACCAATGGATC | TGCGTTGCTTCGGAACGAG |
| | | 408 | (SEQ ID NO: 7) | (SEQ ID NO: 8) |
| AT3G26520 | TIP1-2 | AR- | TTTGTCGCTGTCTCTGTTGG | CCACGGAGGAGAGTGATGTT |
| | | 410 | (SEQ ID NO: 9) | (SEQ ID NO: 10) |
| AT3G16240 | TIP2-1 | AR- | ACTGGGTTGGACCACTCATC | TCGGAAGAACCCATGAAGAC |
| | | 412 | (SEQ ID NO: 11) | (SEQ ID NO: 12) |
| AT4G17340 | TIP2-2 | AR- | TAGGAGCTATTGAAGGAGTAG | ATGAGTCCAGCAAGTGCACCA |
| | | 414 | (SEQ ID NO: 13) | (SEQ ID NO: 14) |
| AT5G47910 | RBOH | AR- | CCGAAGGTCCTTATCGACGG | GTTCTCAATGTCGCTGTCGC |
| | D | 19 | (SEQ ID NO: 15) | (SEQ ID NO: 16) |
| AT1G64060 | RBOH | AR- | TGACACGCCAAGACGAAAGA | GAGCAGAACGAGCATCACCT |
| | F | 21 | (SEQ ID NO: 17) | (SEQ ID NO: 18) |

Primers for ChIP

| | | | | |
|---|---|---|---|---|
| AT1G01620 | PIP1-3 | UTR | TAGCCAACGCAACAGAAT | CATCATCAAGTAGTAGGCTAT |
| | | R1 | (SEQ ID NO: 19) | TAC (SEQ ID NO: 20) |
| | | TSS | GGAAGGGAAAGAAGAGGAT | AGTTCGTGGTCCTTCTAC |
| | | R2 | (SEQ ID NO: 21) | (SEQ ID NO: 22) |
| | | 3OUT | AGTGAATGTTTATGATAAGTTT | GGTAATTTATCTCAAACACAC |
| | | R3 | GT (SEQ ID NO: 23) | AT (SEQ ID NO: 24) |
| AT4G23400 | PIP1-5 | UTR | TGCCTAAATGAACCAAACTAT | AAGTTGGTGGTGATAGAGT |
| | | R1 | (SEQ ID NO: 25) | (SEQ ID NO: 26) |
| | | TSS | CCAACAAGTTCCCAGAGA | ACTATAAGGAACCACCACC |
| | | R2 | (SEQ ID NO: 27) | (SEQ ID NO: 280) |
| | | 3OUT | TCTGTTCCACATGGTAGG | AGCAACTTGTAATATGGATGA |
| | | R3 | (SEQ ID NO: 29) | T (SEQ ID NO: 30) |
| AT3G16240 | TIP2-1 | UTR | ACCACACCACAGAAACTC | AAGGTCTTATCATCTTCTTCAC |
| | | R1 | (SEQ ID NO: 31) | TA (SEQ ID NO: 32) |
| | | TSS | TACAACAATGGCTGGAGT | TTCATCTCCACTTTACTCTTTG |
| | | R2 | (SEQ ID NO: 33) | (SEQ ID NO: 34) |
| | | 5OUT | GAACTAATAATGCAAGTCAAC | AAGATTCGTATAATTCCACCG |
| | | R3 | AG (SEQ ID NO: 35) | (SEQ ID NO: 36) |
| AT4G17340 | TIP2-2 | UTR | CATCTATCATAATTGGTGTGA | GACAACAGAACACTCTTATCT |
| | | R1 | GA (SEQ ID NO: 37) | T (SEQ ID NO: 38) |
| | | TSS | TGGTGAAGATTGAGATAGGAA | CTCTTCTCTTTGTGTTTGCT |
| | | R2 | (SEQ ID NO: 39) | (SEQ ID NO: 40) |
| | | 3OUT | GGAATGAATCGTGTGAGAG | TCTTAACCATAACATTATCCTC |
| | | R3 | (SEQ ID NO: 41) | TC (SEQ ID NO: 41) |
| AT3G61430 | PIP1-1 | UTR | TCTGTCCAAAGTCCAAAC | TTACTACGAAGATAACATTAA |
| | | R1 | (SEQ ID NO: 42) | CAA (SEQ ID NO: 43) |
| | | TSS | AAGATATGGAAGGCAAGGA | GACAACCAATCGGAACATC |
| | | R2 | (SEQ ID NO: 44) | (SEQ ID NO: 45) |
| | | 5OUT | TGTTAGGAGGACATTGGTAA | ATGAATAGCAGCCACAATAA |
| | | R3 | (SEQ ID NO: 46) | (SEQ ID NO: 47) |
| AT4G35100 | PIP2-7 | UTR | TCCAAGGAACATTTAGTAGTT | AAGCAGAATTATGTAGACTAT |
| | | R1 | TA (SEQ ID NO: 47) | ACA (SEQ ID NO: 48) |
| | | TSS | AGAAGTGAGCGAAGAAGG | GTTCATCGCTACACTCCT |
| | | R2 | (SEQ ID NO: 49) | (SEQ ID NO: 50) |
| | | 3OUT | CCCTCTGCTTATCCCTAAA | AACTTGAAACAGACAGCAG |
| | | R3 | (SEQ ID NO: 51) | (SEQ ID NO: 52) |

Chromatin Immunoprecipitation (ChIP) qPCRs

ChIP was conducted as described in previous studies (Shekhawat et al., 2021). Briefly, roughly 1 g of 21-days old mock- and SA190-colonized Arabidopsis grown on ½ MS with or without 25% PEG were cross-linked by vacuum-infiltrating 1% formaldehyde for 15 min and subsequent quenching by 2 M glycine. Nuclei were extracted from the frozen ground powder using NIB (0.4 M Sucrose, 10 mM Tris-HCl pH8, 10 mM MgCl2, 5 mM ß-mercaptoethaol and 1× Proteases Inhibitor cocktail). Nuclei were lysed in NLB (50 mM Tris-HCl pH8, 10 mM EDTA, 1% SDS and 1× Proteases Inhibitor cocktail. Chromatin was sonicated using a Diagenode Bioruptor (40 Hz, 14 cycles each with 30 s on/30 s off with ice cooling), yielding fragments with a size of around 250-350 bp. Antibodies (anti-H3, ab1791; anti-H3K4me3, from Abcam, ab8580 www.abcam.com) were incubated with protein A-coated agarose beads (Invitrogen) for at least 2 hours at 4° C. in IP buffer (1.1% Triton X-10, 1.2 mM EDTA, 16.7 mM Tris-HCl pH8, 167 mM NaCl) and 1× Proteases Inhibitor cocktail. Immunoprecipitations were done in IP buffer at 4° C. for overnight. After washing with low (150 mM NaCl) and high salt (500 mM NaCl) IP buffers and reverse crosslinking, resulted DNA was extracted using the phenol-chloroform method and precipitated with ice chilled ethanol and glycogen (Invitrogen), and then re-suspended in 20 μl of water. ChIP-PCR was performed for three regions of indicated gene loci. Amplification values were normalized to H3 (normalized signal modification/normalized signal H3). The given values in graphs are the means of two biological replicates, with each replicate was normalized to the respective Col-0 with no treatment (mock) sample before averaging.

### Data Availability

The raw data of RNA-sequencing have been deposited in NCBI with GSE Number GSE184355 (ncbi.nlm.nih.gov/geo/query/acc.cgi?acc=GSE184355). Any additional information required to reanalyze the data reported in this study is available from the lead contact upon request.

### Hydrogen Peroxide Staining

To determine H₂O₂ accumulation, Arabidopsis seedlings inoculated or non-inoculated with SA190, grown as described above, were used. Hydrogen peroxide staining of 16-day-old Arabidopsis seedlings with 3,3′diaminobenzidine (DAB) (D5637, Sigma-Aldrich) was performed as described by Ramel, Sulmon, et al. (2009). Briefly, after a gentle vacuum infiltration with 1 mg/ml DAB, dissolved in water (D8001, Sigma-Aldrich), the leaves were incubated in dark for 4-5 hours 618 at room temperature on a shaker at 80-100 rpm. The leaves were then mounted on slides in 30% glycerol and images were taken using a Nikon SMZ25 stereomicroscope.

### Phytohormone Content Analysis

The phytohormones abscisic acid (ABA) and 12-oxo-phytodienoic acid (OPDA) were extracted according to Trapp et al., using a solution of 80% methanol in water containing 10 ng/mL of the internal standards d6-JA and d6-ABA.

The compounds were quantified by HPLC-ESI-SRM on a Thermo Fisher TQS-Altis Triple Quadrupole Mass Spectrometer coupled to a Thermo Scientific Vanquish MD HPLC system. Chromatographic separation was carried out using an UPLC column (Agilent Eclipse Plus C18, RRHD, 1.8 μm, 2.1×50 mm), and the compounds were eluted using water (A) and acetonitrile (B) as the mobile phases at 0.6 mL/min in a gradient elution mode, as follows: 10% B for 0.5 min, 10-55% B at 4.5 min, 55-100% B at 4.7 min, 100% until 6.0 min, 100-10% B at 6.1% and 10% until 8 min. The column was kept at 55° C.

### Field Trials

Open field trials were conducted at the experimental station in Hada Al-Sham (N 21° 47′47.1″ E 39°43′48.8″), Saudi Arabia, in the winter season 2015-2016. The experiment was a randomized complete block design with a split-split plot arrangement of four replicates, plots (2×1.5 m) with seed spacing 20 cm row-to-row. The field was irrigated with 100% or 75% groundwater. The soil had an average pH 7.74 and salinity EC=1.95 dS·m⁻¹. The yield was recorded every 25-30 days from each harvest; and three harvests were done. Field trials data were analyzed as a randomized complete block design, the analysis of variance (one-way ANOVA) of four replicates replications, was performed and LSD (Duncan's test, P<0.05) was calculated to test the significance of differences between means. To inoculate alfalfa (Medicago sativa var. CUF 101) seeds were treated as disclosed in Daur et al., 2018. In brief, a slurry was prepared consisting of sterilized peat, a broth culture of SA190, and sterilized sugar solution (10%) in the ratio 5:4:1 (w/v/v). Subsequently, alfalfa seeds were coated with the slurry at a rate of 50 mL·kg-1. As a control, seeds were coated with a similar mixture without bacteria.

### Greenhouse Experiments

The experiment was carried out at the National Research and Development Center for Sustainable Agriculture (Estidamah) in Riyadh, Saudi Arabia (24.7∘N, 46.7∘E). The glasshouse, 480 m² Venlo type with 8 crop growing gutters, is fully automated with high pressure fogging system, shading screen, PAR sensors and a cooling plastic pad and fan system. Seeds of cucumber (Cucumis sativus cv. Zahran) were washed with water 5 times with water to remove the coated fungicides, after the seeds were mixed with the bacterial solution of SA190 (5.5×10⁷) with 5% sucrose for 3 hours. The seeds were left to dry for an hour at room temperature over three layers of 3 MM paper. The seeds were sown on 20th October of 2020 in germination pots filled with sterilized mixture of peat moss:sand:vermiculite (1:1:1). Three weeks post germination, plants were set on Dutch buckets container filled with clean sand (16 kg per container) as a soilless culture system. For the pots irrigation 50 ml (1.25×10⁸) of SA190 solution per plant were used every 20 days for 6 different irrigation doses during the plant growth. All plants were grown on a high wire system with a 3.75 plants per m². The mature cucumber fruits were harvested twice a week to calculate total yield as kg/m². The climate and fertigation control as well as the data collection were arranged through a greenhouse HortiMaX MultiMa control unit.

For irrigation treatments, plants were divided into two groups: well irrigated (100% irrigated) and water stressed (70% irrigated). Plants received during the growing season (150 days) 625 L/m² for 100% irrigation and 70% (435 L/m²) for water stress treatment. Water use efficiency was calculated with yield as the agnomical base. Water Use Efficiency=(Total water/total yield). Under 100% irrigation, non-treated plants produced fruit required 22.7 L/kg, while SA190-treated plants 21 L/kg of water. Under 70% irrigation the yield was 18.9 L/kg for non-treated and 17 L/kg for SA190-treated plants.

The average day/night glasshouse temperature was 25 and 18° C., respectively, with a 70-80% relative humidity. However, pH and EC of the nutrient solution were monitored regularly according to the commercial production requirements. Greenhouse experiments were arranged in a randomized block de-sign based on a factorial experiment. Analysis of variance (one-way ANOVA) of three replications, was performed and LSD (Duncan's test, P<0.05) was calculated to test the significance of differences between means.

### Results

Pseudomonas argentinensis Strain SA190 Enhances Drought Tolerance of Arabidopsis and Crops in Desert Agriculture

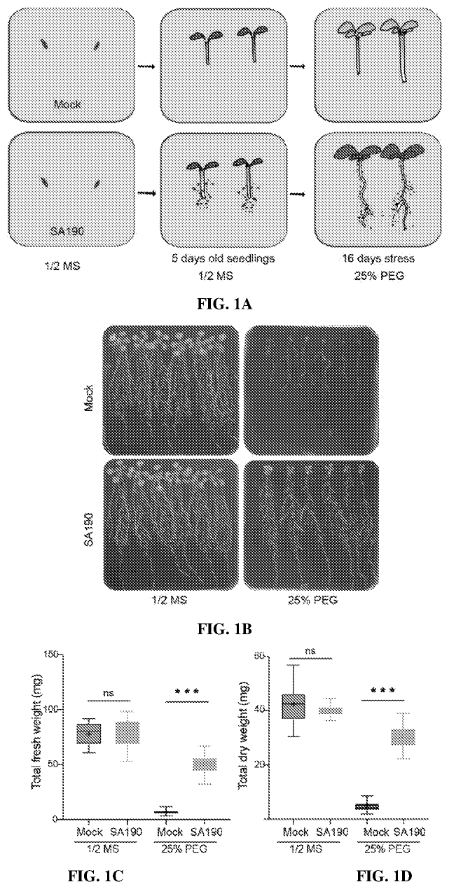

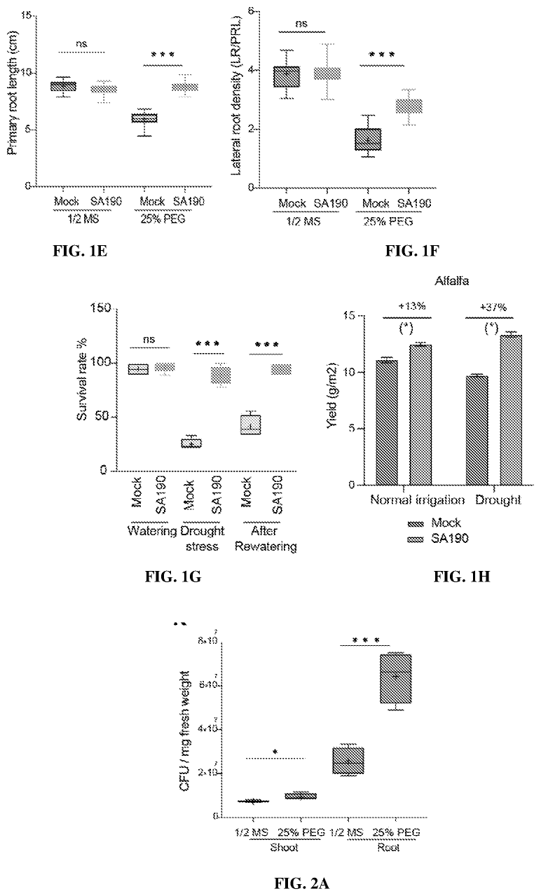

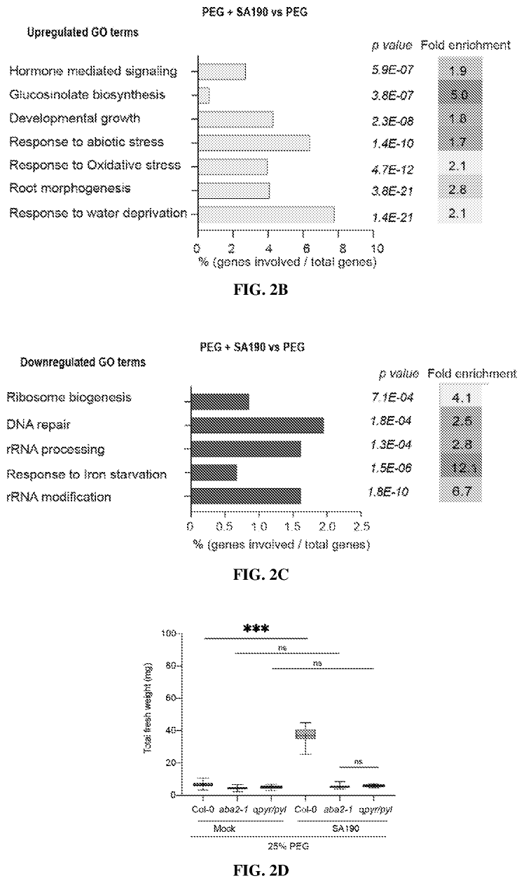

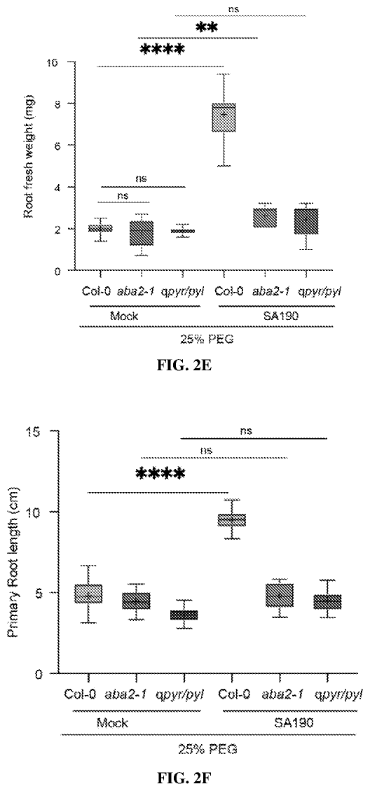

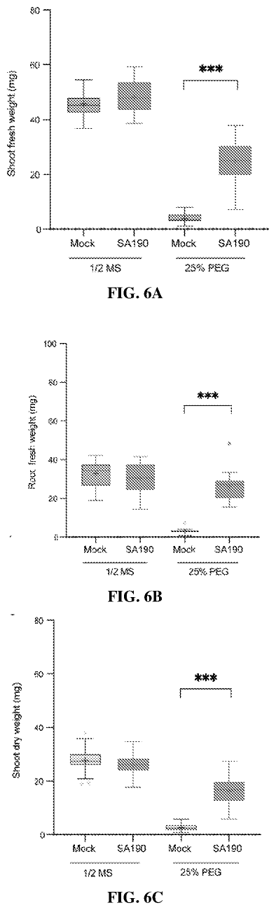

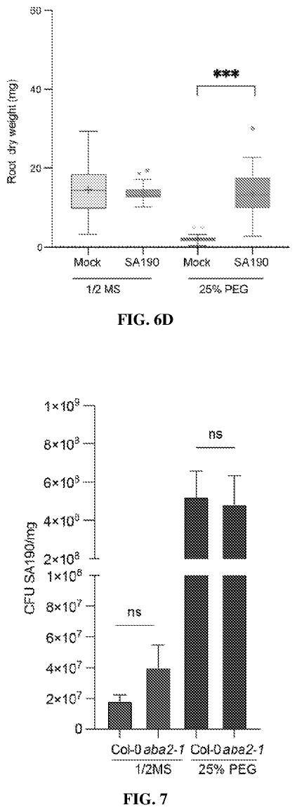

Pseudomonas argentinensis strain SA190 is a plant endophytic bacterium that was previously isolated from root nodules of the desert plant Indigofera argentea (Lafi et al., 2016). To test the effect of SA190 under drought mimicking conditions, plants were germinated for 5 days on ½ MS agar medium containing 10⁸ cfu of P. argentinensis strain SA190 before transfer to fresh ½ MS plates infiltrated with 0 or 25% PEG (FIG. 1A). After 16 days, plant morphology, total fresh and dry weight, root length and lateral root density were determined (FIGS. 1A-2A). The results show that SA190 did not significantly influence the morphology or development of Arabidopsis under non-stress conditions (FIG. 1B). Upon 25% PEG treatment, the growth of non-colonized plants was severely inhibited (FIG. 1B) resulting in a reduction of fresh and dry weight by more than 90% (FIGS. 1C-1D). In contrast, Arabidopsis colonized with SA190 maintained growth, resulting in a 6-fold enhancement of fresh and dry weight on 25% PEG (FIGS. 1C-1D, FIGS. 6A-6D). Also, a significant increase in the primary root length and development of the lateral root system was observed in SA190 colonized plants (FIGS. 1E-1F).

To see the effect of SA190 on the growth of adult plants under drought conditions, 5 day-old non-colonized or SA190-colonized Arabidopsis were transferred to pots for two weeks. Thereafter, watering was stopped, and plants were subjected to drought stress for three weeks. While non-colonized (Mock) plants showed a drop in survival by more than 70%, SA190-colonized plants were affected by less than 10% (FIG. 1G and data not shown). One week after re-watering, less than 50% of non-colonized plants resumed growth in comparison to a 100% of SA190-colonized plants (FIG. 1G and data not shown). These results indicate that SA190 significantly enhances drought stress tolerance of Arabidopsis plants.

To evaluate the agronomic potential of SA190 on crops, we performed growth room experiments with alfalfa which is used as an important animal feed in different regions of the world. Alfalfa seeds were coated with SA190 and tested in parallel with mock-coated seeds in control growth room conditions. A randomized pot experiment with different replicates was used with a drought protocol. After 10 days of drought (no irrigation), SA190-inoculated alfalfa plants produced 13% more biomass than non-inoculated plants under a full irrigation regime (FIG. 1I). When the plants were exposed to 10 days drought stress (no irrigation), the SA190-inoculated plants showed 37% more biomass under the drought treatment compared to non-inoculated plants. (FIG. 1I). SA190 enhances Alfalfa drought stress tolerance. Plants were mock- or SA190-inoculated and grown for 42 days. Plants were then further irrigated (upper panel) or irrigation was stopped for 10 days (drought) before analysis. (data not shown). These results show that SA190 can efficiently enhance the performance of alfalfa under drought conditions.

### SA190-Enhanced Root Architecture and Colonization of Arabidopsis Upon PEG Drought Stress Proxy

Since SA190 was isolated from roots of the desert plant Indigofera argentea, the interaction between SA190 and Arabidopsis was characterized in more detail. After 16 days on normal or 25% PEG medium, root length and lateral root density were determined. The results show that SA190 did not significantly influence the morphology or development of Arabidopsis roots under non-stress conditions (FIG. 1B, FIG. 6B, FIG. 6D). However, on 25% PEG medium and in contrast to non-colonized plants, SA190-inoculated plants maintained their root development at WT level while mock-treated plants had a strong reduction in root growth (FIG. 1B), with a massive increase in root fresh and dry weight (FIG. 6B, FIG. 6D) mostly due to enhanced growth of the primary and development of the lateral root system (FIGS. 1E-1F).

The root colonization by SA190 was investigated. To this end, SA190 was stably transformed with a GFP expressing cassette (SA190: GFP). Confocal microscopy revealed that SA190: GFP mainly colonized roots (data not shown), preferentially epidermal cells of the root differentiation zone (data not shown). These findings were confirmed by determining the SA190 colony forming units (CFU) of root and shoot tissues (data not shown). Interestingly, the roots of plants grown on 25% PEG showed enhanced colonization when compared to plants grown only on ½ MS agar (data not shown). At later stages of colonization, SA190: GFP was also found inside of root tissues revealing the endophytic nature of SA190 (data not shown).

### SA190 Reprograms the Transcriptional Response of Arabidopsis to PEG Stress

To understand the molecular mechanism of SA190 induced PEG tolerance, RNA-seq analysis was performed of 21-day old non- or SA190-colonized roots upon normal and PEG stress for 16 days. The root transcriptome data were organized by hierarchical clustering into groups of differentially expressed genes (DEGs) according to their expression patterns in non-colonized (Mock) and SA190 colonized plants (SA190) under normal conditions, as well as non-colonized and SA190-colonized plants under PEG stress conditions, denoted as PEG and PEG+SA190, respectively (data not shown). The gene ontology enrichment of the up- and down-regulated root DEGs was assessed. When comparing SA190- to mock-colonized plants, observed were 30 up- and 145 down-regulated DEGs under non-stress conditions. In this relatively small gene set, nonetheless found was significant GO term enrichment of genes related to cell wall organization (e.g. expansins and pectin lyases) and response to water deprivation.

Under PEG stress conditions, massive changes in DEGs were seen between SA190-colonized and non-colonized roots revealing 2384 up and 1365 down-regulated genes. The up-regulated genes showed enrichment in responses to hormone signaling, glucosinolate biosynthesis, water deprivation and oxidative stress as well as root morphogenesis and developmental growth (FIG. 2B). The most significant GO terms in the set of down-regulated genes were related to ribosome, RNA metabolism and DNA repair (FIG. 2C). Interestingly, the hormone, abiotic stress and water deprivation-related GO terms were all related to ABA signaling, including the ABA receptor components PYL1, PYL3, the ABA protein phosphatases HAI1, and HAI2, as well as the key ABA responsive protein kinase SnRK2-3 and a large number of aquaporin genes.

### ABA Pathway Mutants are Compromised in SA190-Induced PEG Stress Tolerance

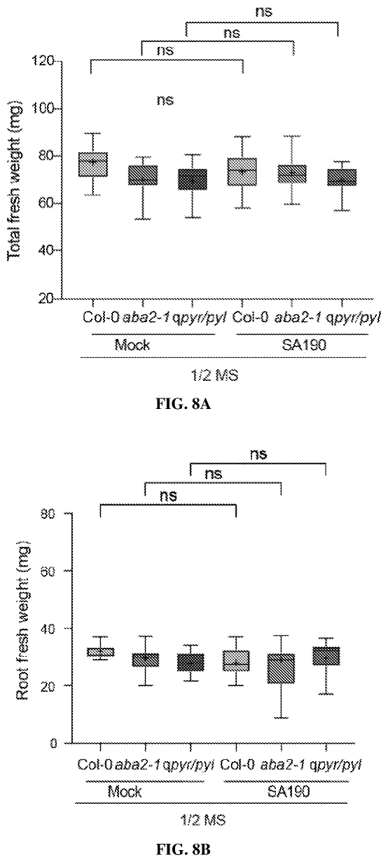

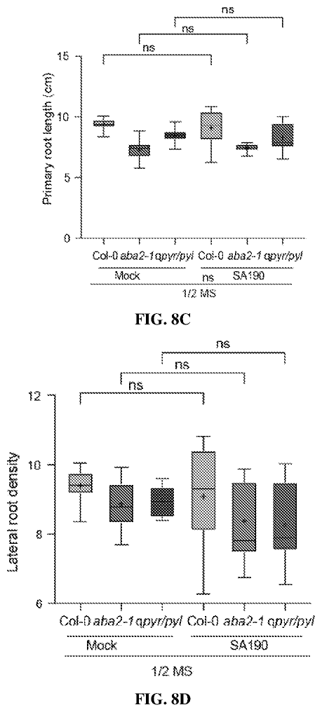

Since ABA plays a key role in drought stress tolerance and our transcriptome analysis showed that SA190 colonization of plants affects ABA signaling under PEG stress, Arabidopsis mutants impaired in ABA biosynthesis (aba2-1) and signaling pyr1 pyl4 pyl5 pyl8 (qpyr/pyl) were tested. Under non-stress conditions, no significant differences were observed in the fresh or dry weight of SA190-colonized and non-colonized plants between WT and both mutants (FIG. 8A). Under PEG drought proxy stress, however, SA190-maintained plant growth was strongly compromised in both aba2-1 and qpyr/pyl mutants (FIG. 2D), indicating a central role of the ABA pathway in the SA190-induced Arabidopsis PEG stress tolerance.

### SA190-Induced Changes in Root Architecture are Dependent on ABA Signaling

Since ABA plays an important role in pathogenic plant-microbe interactions, it was tested whether ABA-deficient mutants might compromise the interaction of Arabidopsis with SA190 and therefore result in the loss of the beneficial effect on drought stress tolerance. However, similar levels of SA190 colonization were seen under non-stress (½ MS) and drought proxy stress (½ MS+25% PEG) conditions (FIG. 7), indicating that ABA does not influence SA190 colonization of Arabidopsis.

Since SA190 induces massive changes in Arabidopsis root structure, whether ABA pathway mutants affect SA190-induced modification of Arabidopsis root architecture was tested. Under non-stress conditions, no significant differences were observed in the fresh weight of SA190-colonized and non-colonized roots when comparing WT to either ABA biosynthesis (aba2-1) or ABA signaling pyr1 pyl4 pyl5 pyl8 (qpyr/pyl) mutants (FIGS. 8B-8D). Under PEG drought proxy stress, however, the SA190-induced changes in root structure, including fresh weight, primary root length and lateral root density were strongly compromised in both aba2-1 and qpyr/pyl mutants (FIGS. 2E-2G), indicating a central role of the ABA pathway in SA190-induced changes in root architecture.

### SA190 Modulates the Expression of Aquaporin Genes Under PEG Drought Proxy Stress in an ABA-Dependent Manner

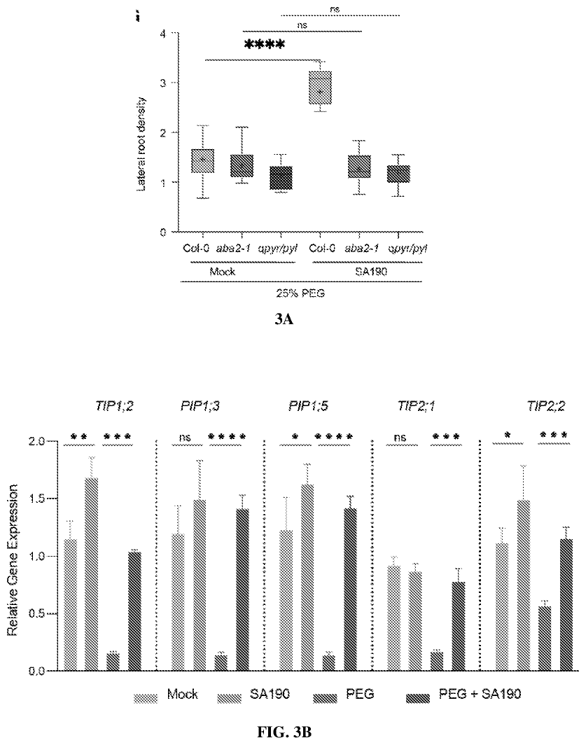

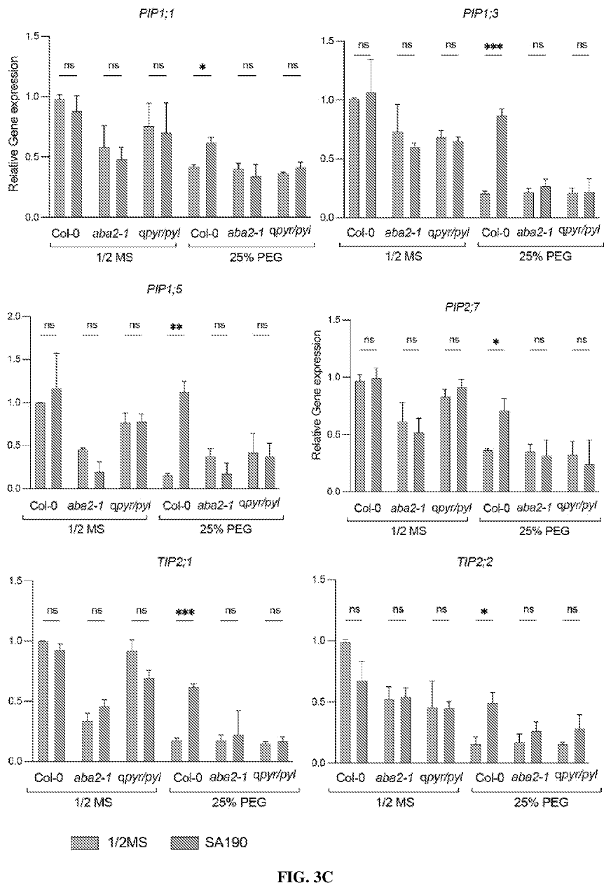

RNAseq analysis of root tissue revealed that under PEG conditions, a large set of Arabidopsis genes including several aquaporins were differentially regulated by SA190 (data not shown). Since aquaporins are critical elements in adjusting the water flow in physiologically critical situations (Li et al., 2014), many of which are regulated by ABA (Pawlowicz & Masajada, 2019), these aquaporin genes were analyzed as proxy for the set of SA190-regulated genes. Under non-stress conditions, roots of SA190-colonized compared to non-inoculated plants (FIG. 3B), qRT-PCR analysis of aquaporin transcript levels changed only mildly and in most cases not in a statistically significant manner. However, under PEG stress, SA190-colonized roots showed massively enhanced expression compared to roots of non-colonized plants (FIG. 3B). The influence of the ABA biosynthesis and ABA signaling pathways on SA190 induced aquaporin gene expression was tested. Most aquaporin genes did not show significant differences in expression under non-stress conditions in either aba2-1 or qpyr/pyl mutants (FIG. 3C). Under PEG-stress conditions, however, the two types of ABA mutants strongly compromised the SA190-enhanced expression of all tested aquaporin genes (FIG. 3C), indicating that the ABA pathway is essential in mediating the SA190-induced changes of aquaporin gene expression under PEG-stress.

### Epigenetic Priming of SA190-Targeted Genes is Mediated by the ABA Pathway

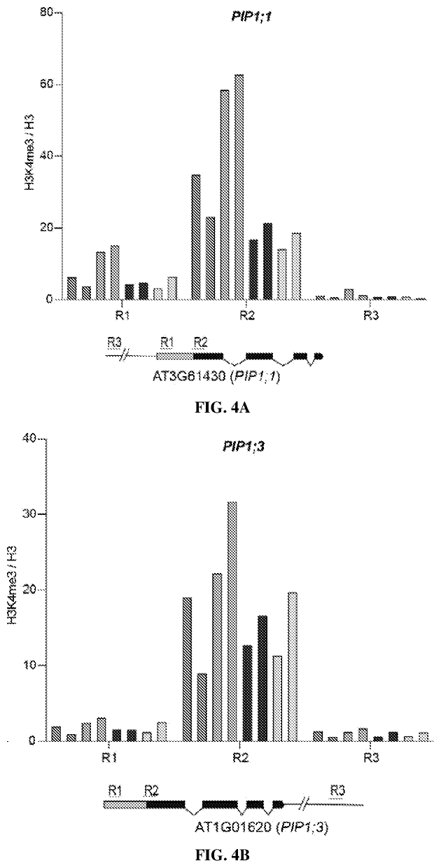

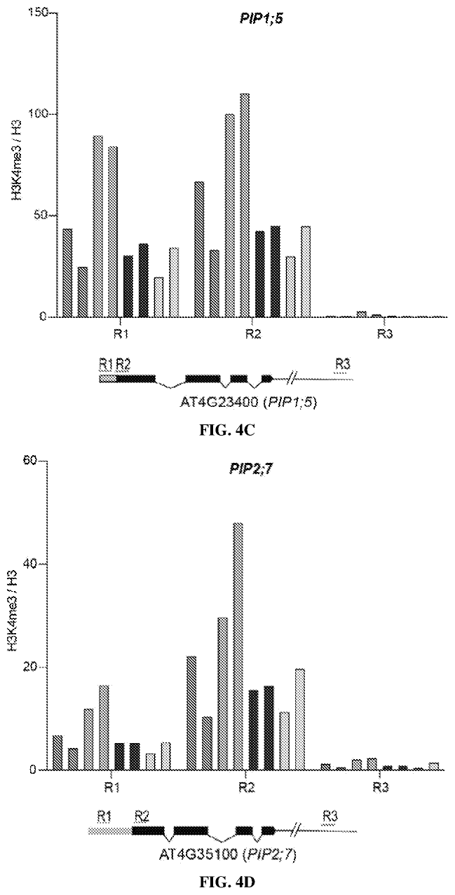

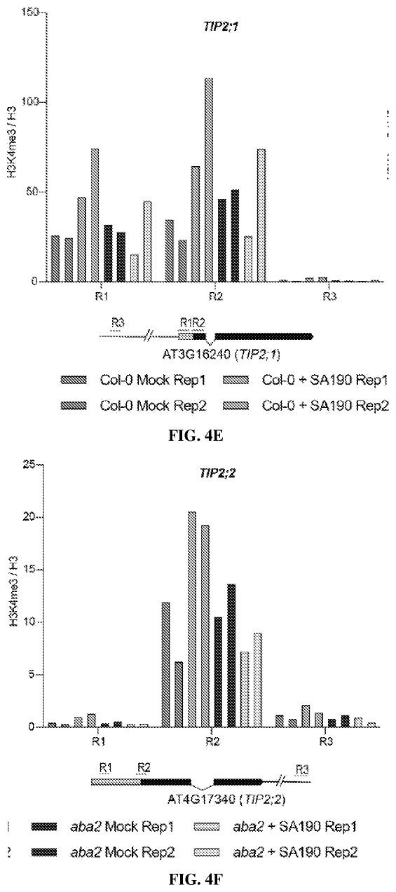

The transcriptome analysis of SA190-colonized plants showed that the expression of genes is not constantly increased, but only shows differential changes under drought stress proxy conditions. Whether SA190 colonization might already be related to changes in the epigenetic state of the differentially regulated aquaporin genes under non-stress conditions was tested. As described in FIGS. 1A-2A, plants were germinated on SA190 containing ½ MS media for 5 days before transfer onto bacteria-free ½ MS+25% PEG. As control, plants that were germinated in parallel but in the absence of SA190 (mock) were used. As H3K4me3 was found as a priming mark for genes in drought-trained plants (Ding et al, 2012a), different regions of the set of SA190-regulated aquaporin genes for H3K4me3 enrichment were tested. A significant enrichment of the H3K4me3 mark in these aquaporin genes were observed, especially near transcription start sites (FIG. 4A). Since ABA was essential for mediating SA190-induced aquaporin gene expression and PEG drought stress proxy tolerance, the epigenetic status of these aquaporin genes in the ABA-deficient aba2-1 mutants were also analyzed. In non-colonized (mock) plants, the aba2-1 mutant showed similar levels of H3K4me3 as wild type plants. In SA190-colonized plants, however, aba2-1 mutant plants were completely compromised in the enhancement of H3K4me3 levels in the aquaporin gene loci. It was concluded that SA190 primes aquaporin genes in an ABA-dependent manner by enhancing H3K4me3 levels.

### Role of Aquaporins in SA190-Induced PEG Drought Stress Proxy Tolerance

The analysis suggested that aquaporins might be key targets and mediators of SA190-induced drought stress tolerance. Water movement across the cellular membranes is largely regulated by a family of water channel proteins called aquaporins. Aquaporins play a key role in transmembrane water transport in environmental stress responses (Li et al., 2014; Maurel et al., 2015) and their enhanced expression increased the root osmotic hydraulic conductivity, transpiration and shoot to root ratio while the downregulation lead to higher drought stress susceptibility (Pawlowicz & Masajada, 2019). In Arabidopsis there exist 35 aquaporin genes (Johanson et al, 2001) which can be broadly divided into plasma membrane intrinsic proteins (PIPs) and tonoplast intrinsic protein (TIPs) genes. Some of the Arabidopsis PIPs and TIPS proved to be active water channels in Xenopus oocytes (Quigley et al, 2002). The biological significance of aquaporins in plants is their ability to modulate transmembrane water transport in situations where adjustment of water flow is physiologically critical (Li et al, 2014). PIPs play an important role in controlling the transcellular water transport and are subdivided into the two subfamilies PIP1 and PIP2 (Maurel et al, 2015). The overexpression of PIP isoforms increased the root osmotic hydraulic conductivity, transpiration and shoot to root ratio while the downregulation leads to drought stress susceptibility (Pawlowicz & Masajada, 2019). However, when expressed in a heterologous systems, the overexpression of aquaporins can lead to a negative effect on stress resistance, due to the fact that the native stress response machinery may recognize them as a foreign proteins (Li et al., 2014).

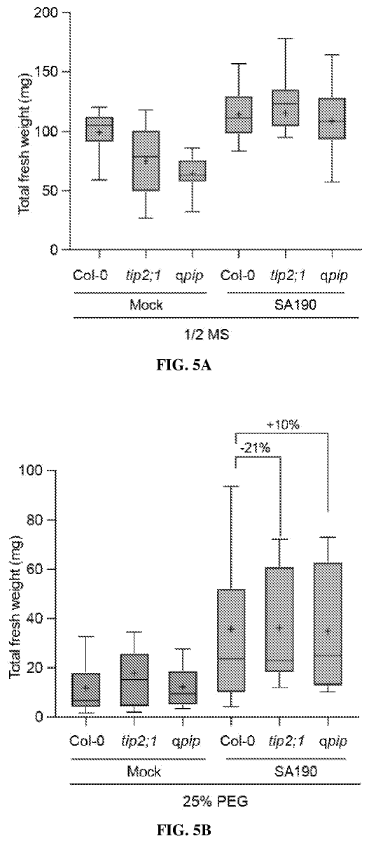

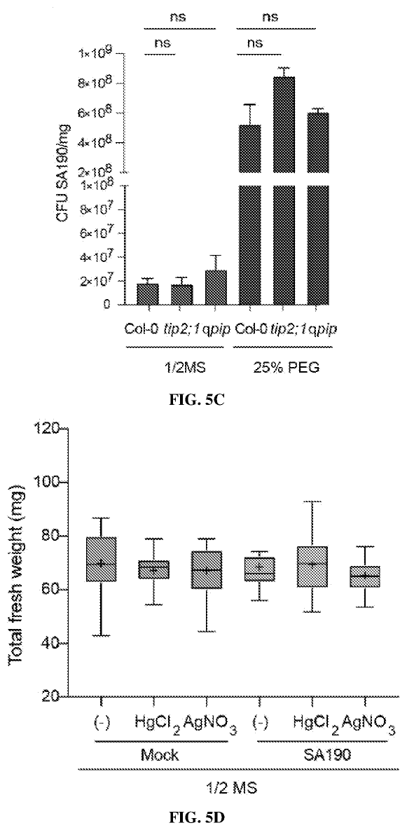

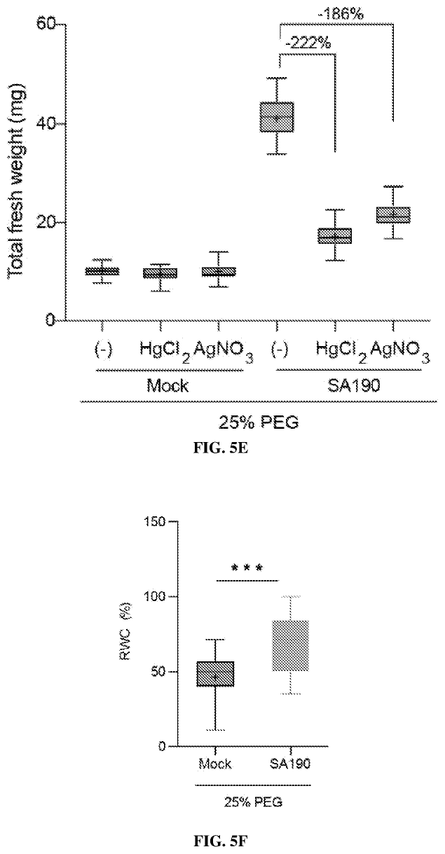

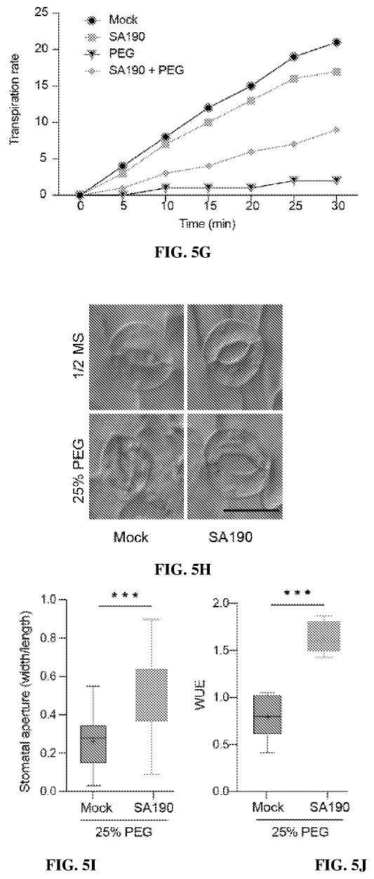

To directly test the significance of the set of aquaporins identified in the SA190 transcriptome under PEG stress conditions, the quintuple pip1;1 pip1;2 pip1;3 pip1,4 pip1;5 (qpip) and tip2;1 Arabidopsis mutants in the absence and presence of SA190 were used (FIGS. 5A and 5B). No significant changes of SA190-induced PEG drought proxy tolerance were observed when comparing growth of WT with qpip and tip2;1 mutants under non-stress (FIG. 5A) or PEG stress conditions (FIG. 5B). Also assessed was whether the aquaporin mutants affect SA190 colonization. Although PEG conditions clearly induced SA190 colonization of Arabidopsis, this effect was similar for WT, qpip and tip2;1 mutants (FIG. 5C).

The maintenance of the beneficial response of Arabidopsis to SA190 in the pip and tip mutants might also be due to the redundancy of the large aquaporin gene family in Arabidopsis. To overcome this problem, two known aquaporin inhibitors, AgNO₃ and HgCl₂, were used; these inhibitors had no effect on Arabidopsis growth under normal conditions, irrespectively whether the plants were non- or SA190-colonized (FIG. 5D). However, under PEG drought stress proxy conditions, the inhibitors completely compromised the beneficial effect of SA190 on Arabidopsis (FIG. 5E).

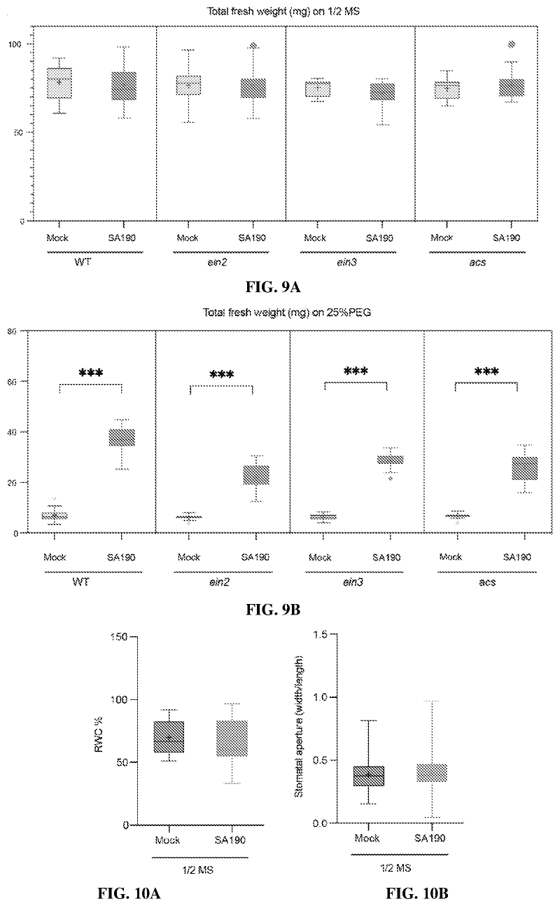

The fact that one of the aquaporin inhibitors was AgNO₃, which is also an inhibitor of ethylene signaling, raised the question of its role in the beneficial interaction of SA190 with Arabidopsis. A number of ethylene biosynthesis and signaling mutants were tested for a possible role in the SA190-Arabidopsis interaction. None of the ethylene mutants affected the growth of SA190-colonized plants in comparison to non-colonized plants under non-stress conditions (FIG. 9A). Compared to the massive loss of beneficial activity of SA190 in ABA mutants (FIG. 2D), SA190-induced PEG drought stress proxy tolerance in the ethylene mutants was only mildly affected under water limiting conditions (FIG. 9B), suggesting that ethylene does not play a major role in mediating SA190 PEG stress tolerance of Arabidopsis plants.

### SA190 Colonization Changes Plant PEG Drought Stress Proxy Physiology

The importance of aquaporins in regulating transpiration and photosynthesis in drought stress and its importance in water use efficiency, growth and yield was recently highlighted (Moshelion et al, 2015). Whether similar effects could be observed in SA190-colonized plants when exposed to PEG stress conditions was tested.

Since drought stress leads to a decreased water content in plants, the water content of leaves in SA190-colonized and non-colonized plants under non-stress and PEG drought proxy conditions were measured. Under non-stress conditions, no significant differences in the relative water content (RWC) of leaves were observed when comparing SA190- and non-colonized plants (FIG. 10A). PEG stress resulted in the reduction of the relative leaf water content in non-colonized plants (FIG. 5F), but not in SA190-colonized plants, which maintained leaf relative water content to the same level as seen in plants grown under non-stress conditions (FIG. 10A).

Plants commonly react to drought stress by reducing transpiration to save water. However, reduced transpiration also means reduced photosynthesis and growth. Under non stress conditions, a slightly lower transpiration rate in SA190-colonized plants was observed when compared to non-colonized plants (FIG. 5G). In contrast, whereas growth on 25% PEG resulted in a reduced transpiration rate in non-colonized plants, SA190-colonized plants showed a much higher transpiration rate, consistent with a better overall water status under stress conditions (FIG. 5G).

Stomatal closure is a common adaptation response of plants to drought conditions to reduce water loss. Stomatal opening under non-stress and 25% PEG in SA190-colonized and non-colonized plants (FIG. 5H). Under non-stress growth conditions, no statistically significant differences in stomatal aperture were observed between SA190-colonized and non-colonized plants (FIG. 10B). However, under drought stress proxy conditions, non-colonized plants rapidly induced stomatal closure, whereas SA190-colonized plants showed significantly more open stomata (FIG. 5I).

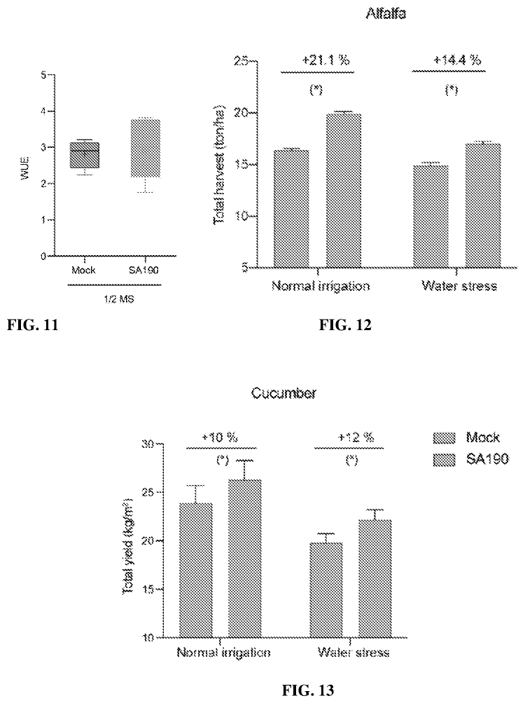

The observation that SA190-colonized plants showed higher water contents and significantly higher growth rates under water limiting conditions suggested that SA190 plants might make better use of the available water. Whether SA190 altered the water use efficiency (WUE) of its host plant was assessed. No significant changes in water use efficiency between mock- and SA190-colonized plants under normal conditions were observed (FIG. 11). However, when plants were exposed to PEG drought stress proxy conditions, the water use efficiency of SA190-colonized plants was clearly superior to that of non-colonized plants (FIG. 5J). Overall, these results suggest that an enhanced water status in SA190-colonized plants is the basis for maintaining growth of Arabidopsis plants under limiting water conditions.

### SA190 Modulates Arabidopsis Phytohormone and Camalexin Levels Under Drought Stress

Abscisic acid (ABA) is one of the key phytohormones that regulates the plant response to drought stress. ABA acts through a conserved signal transduction pathway, comprised of a PYRABACTIN RESISTANCE 1-Like (PYL)-PROTEIN PHOSPHATASE 2C (PP2C) and SNF1-RELATED PROTEIN KINASE 2 (SnRK2) module. ABA binding to PYL protein triggers a conformational change in receptors which allows it to bind and inhibit the PP2C that normally represses ABA signaling. This PYL-ABA-PP2C complex releases SnRK2 from the otherwise inhibitory complex with PP2C, initiating phosphorylation of transcription factors which regulate gene expression involved in ABA output responses (Chen et al, 2020; Cutler et al, 2010; Fujii et al, 2009). ABA also regulates the early production of reactive oxygen species (ROS) after drought perception (Cruz de Carvalho, 2008). ROS serves as a stress signal to activate downstream processes, but accumulation of ROS can lead to cell damage and growth penalty.

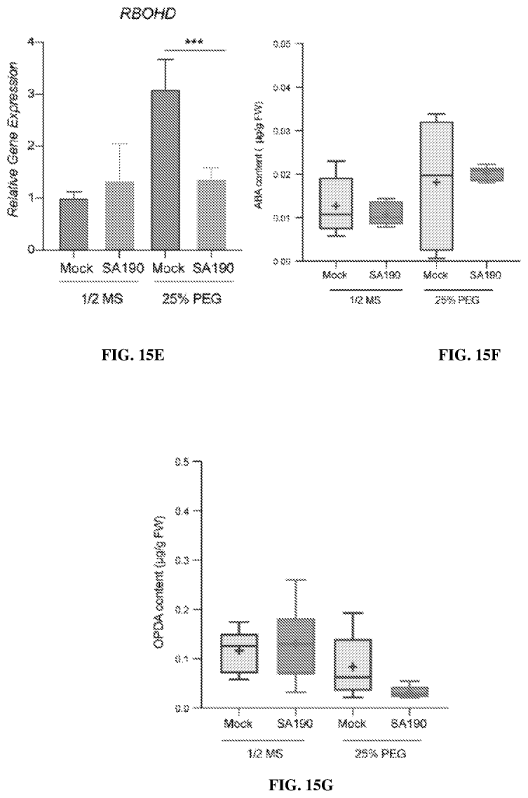

Since the transcriptome analysis indicated possible roles of several hormone pathways in the SA190-induced growth promotion under drought stress, the levels of the key drought-related plant hormone abscisic acid (ABA) from SA190- and non-colonized plants were measured. Under normal conditions, there was no statistically significant difference of ABA contents between SA190- and non-colonized shoots and roots (FIGS. 15A, and 15F. In non-colonized shoots, ABA levels strongly increased upon drought stress (FIG. 15A); ABA levels increased to a lower degree in SA190-colonized plants. In roots, ABA levels also increased under drought conditions, but to the same level in colonized and non-colonized plants.

OPDA levels were determined as a proxy for JA. Under normal conditions, OPDA levels were slightly higher in SA190-colonized compared to control plants. Drought stress enhanced OPDA levels in shoots but decreased them in roots. SA190-colonization of plants under drought stress resulted in suppression of the shoot OPDA levels, but to a further decrease in roots (FIGS. 15B, and 15G).

Taken together, the shoot data suggest that SA190 actively alters levels of several plant hormone levels related to drought tolerance.

### SA190 Regulates Reactive Oxygen Species Biosynthesis

Generation of ROS is one of the major and key responses in plants under drought stress, which cause oxidation of cellular components and thus leading to severe cellular damages (Kar, 2011, Wang et al., 2012). To detect the H₂O₂ levels in SA190 colonized and non-colonized plants, the leaves of 16 d old PEG stressed seedlings were stained with DAB (3, 3′-Diaminobenzidine). Under normal conditions, the WT and SA190 colonized plants showed similar staining, thus showing no differences in H₂O₂ production. However, after growth on PEG, SA190-colonized plants showed a substantial decrease in H₂O₂ accumulation, as seen by reduced DAB staining in leaves compared to non-colonized control plants (FIG. 15C). Respiratory burst oxidase homologues (RBOHs) are critical enzymes that produce ROS in response to environmental signals in plants (Kwak et al., 2003). We observed a lower expression of two of the key ROS producing RBOHs namely RBOHD and RBOHF in SA190 colonized plants after PEG stress (FIGS. 15D and 15E). These results suggest that SA190 colonized plants produce less ROS during PEG stress than the non-colonized ones.

### SA190 Maintains Crop Growth Under Reduced Irrigation Conditions in the Field

The potential agronomic use of SA190 as a biological solution to crop plants under real agricultural conditions both on the open field and under greenhouse conditions was evaluated. For open field trial, the beneficial activity of SA190 on growth parameters of the multi harvest crop plant alfalfa (Medicago sativa), which is largely used as animal feed in different regions of the world, was tested. Alfalfa seeds were coated with SA190 and tested in parallel with mock-coated seeds in an experimental field station near Jeddah, Saudi Arabia. A randomized complete block design with a split-split plot arrangement with different replicates was used on the growth seasons (2015-2016) with 100 or 70% water irrigation. SA190-inoculated alfalfa plants exhibited an increase in yield by 21.1% in normal irrigation condition and 14.4% under water stress conditions (FIG. 12). In terms of greenhouse production conditions, the effect of SA190 was evaluated on cucumber grown in pot sand culture. SA190-treated plants increased the total yield by 10% under full irrigation conditions (FIG. 13).

Subsequent studies assessed the effect of SA190 on the productivity of these two crops under limited water irrigation. A reduction of water irrigation by 30 and 25% decreased alfalfa and cucumber production by 9.4% and 17.1%, respectively. In comparison, SA190-colonized alfalfa and cucumber enhanced productivity by 14.4% and 12%, respectively (FIGS. 12 and 13). These results show that SA190 can efficiently enhance the yield of two important crops in open field and greenhouse production conditions.

Discussion Water is critical for life on earth and is a major determinant of food production worldwide. Due to the increasing world population and climate change, water is becoming increasingly scarce on our planet. Since globally 70% of all freshwater is used by agriculture, increasing the water use efficiency of crops as well as making crops more drought tolerant are among the most important targets in agricultural engineering. Since water uptake primarily occurs via roots, strategies to improve water use efficiency through changes in root system architecture have recently gained an increased interest. Besides genetic engineering and molecular breeding, the application of PGPBs might offer another way to modify the root system and enhance water use efficiency and drought tolerance of crops. This study reports that the root endophytic bacterium Pseudomonas argentinensis strain SA190 isolated from the roots of the desert plant Indigofera argentea induces major changes in root architecture and maintains growth of plants under limiting water conditions. Although SA190 was isolated from root nodules, it is unlikely that the enhanced plant drought tolerance is due to enhanced nitrogen fixation by SA190. First, analysis of the SA190 genome did not reveal any of the known nif genes (nifHDK) involved in nitrogen fixation nor the nodABC genes encoding the biosynthesis enzymes for synthesis of nodulation factors (Lafi et al., 2016). Moreover, it was not observed that SA190 could enhance growth of Arabidopsis on N-free or N-limiting conditions. It was therefore concluded that N-fixation is not part of the mechanism how SA190 induces drought tolerance in plants.

Tolerance to environmental stresses can be achieved via different modes of cellular and biochemical changes conferred by beneficial microbes. Among the physiological and molecular responses to drought conditions, the plant phytohormone ABA plays a central role in drought adaptation of plants. ABA levels rapidly rise upon water limitation, resulting in stomatal closure to reduce transpiration and save water at the cost of growth (Zhu, 2002). Interestingly, under drought stress proxy conditions, SA190-colonized plants showed an improved leaf water status and transpiration rate as well as maintaining plant growth. These experiments were conducted with the genetic model plant Arabidopsis under maximally controlled growth conditions. To test whether SA190 might also be able to enhance drought tolerance to a crop plant, drought stress experiments were performed on the important fodder plant alfalfa. SA190-colonized alfalfa showed significantly enhanced drought resilience than non-colonized plants. These results support the concept that beneficial microbes such as SA190 might be a powerful tool to sustain agriculture production during drought events.

The analysis of the interaction between SA190 and Arabidopsis in the context of limited water availability revealed many of the known players of drought stress tolerance in plants. In particular, many genes involved in the ABA pathway were found and subsequent genetic analysis showed that ABA is essential for mediating SA190-induced PEG drought stress proxy tolerance. Transcriptome and qRT-PCR analysis showed that at least seven aquaporin genes are differentially regulated in SA190-colonized plants under PEG conditions and this regulation by SA190 was found to be entirely dependent on the ABA pathway. Because aquaporins play a key role in adjusting water relations in plants and many are regulated by ABA (Jang et al, 2004), these genes were used as a proxy for the set of SA190-regulated ABA-dependent genes. Intriguingly, expression of all identified aquaporin genes was strongly suppressed under PEG drought stress proxy conditions in non-colonized plants, but none of these aquaporin genes were found to have significantly altered expression under non-stress conditions in SA190-colonized or non-colonized plants. These results suggested a potentially epigenetic priming mechanism. Since H3K4me3 enrichment is associated with transcriptionally active gene regions of drought responsive genes (van Dijk et al, 2010), the set of differentially regulated aquaporin genes in the context of SA190 colonization were investigated. It was found that SA190 primes aquaporin genes by enhancing H3K4me3 levels in the regions near the transcriptional start sites. Priming by SA190 occurs already in the absence of PEG drought stress conditions. Therefore, SA190 interaction functions via priming genes for enhancing their expression under water limiting conditions. The H3K4me3 mark is broadly distributed on many ABA inducible genes (van Dijk et al., 2010) and mutants in the key enzyme of the ABA biosynthesis pathway NCED3 show enriched H3K4me3 levels under drought stress conditions (Ding et al, 2011). This modification is mediated by the histone methyl transferase ATX1 (Arabidopsis trithorax-like 1), as transcript levels of several ABA and drought-upregulated genes like RD29A and RD29B were reduced during drought treatment in the atx1 mutant (Ding et al, 2012b). It will be interesting to see if ATX1 and/or other methyl transferases are also involved in mediating H3K4me3 priming of SA190-targeted genes.

Aquaporins play a key role in transmembrane water transport in transpiration and environmental stress responses (Maurel et al., 2015). In Arabidopsis there exist 35 aquaporin genes (Johanson et al., 2001) which can be broadly divided into plasma membrane intrinsic proteins (PIPs) and tonoplast intrinsic protein (TIPs) genes. Some of the Arabidopsis PIPs and TIPs proved to be active water channels in Xenopus oocytes (Quigley et al., 2002). The biological significance of aquaporins in plants is their ability to modulate transmembrane water transport in situations where adjustment of water flow is physiologically critical (Li et al., 2014). PIPs play an important role in controlling the transcellular water transport and are subdivided into the two subfamilies PIP1 and PIP2 (Maurel et al., 2015). The overexpression of PIP isoforms increased the root osmotic hydraulic conductivity, transpiration and shoot to root ratio while the downregulation leads to drought stress susceptibility (Pawlowicz & Masajada, 2019). However, when expressed in heterologous systems, overexpression of aquaporins can also have negative effects on stress resistance, due to the fact that the native stress response machinery may recognize them as a foreign protein (Li et al., 2014). Aquaporins are also regulated by post-translational modification including phosphorylation, methylation, acetylation, glycosylation, and deamination. Although fine-tuning aquaporin expression plays a key role in all functions related to the water status of plants, the multitude of genes and their potential redundancy makes genetic analyses a complicated matter. Redundancy of aquaporins in Arabidopsis might also be due to limiting our success in analyzing aquaporin mutants with respect to SA190-induced tolerance to PEG drought stress proxy (FIG. 5B). However, using aquaporin inhibitors (AgNO₃ and HgCl₂), although probably not highly specific, independently suggested that aquaporins might mediate part of SA190-induced PEG stress tolerance (FIG. 5D).

Drought stress massively alters many physiological parameters in plants, with ABA playing one of the primary roles in abiotic stress tolerance (Nakashima & Yamaguchi-Shinozaki, 2013; Zhu, 2002). A cluster of ABA-related genes was found in the transcriptome of SA190-colonized plants. The genetic analysis showed that the ABA pathway is essential for mediating SA190-induced PEG drought stress proxy tolerance by priming of target genes. The transcriptional responsiveness of drought stress regulated genes is correlated with changes in histone modifications (Kim et al, 2012; Kim et al, 2008; To & Kim, 2014). Two of the important histone modifications, H3K4me3 and H3K9ac, are enriched on drought stress-regulated genes like RD20 and RD29A. The levels of these modifications change from mild to severe drought stress, suggesting that epigenetic responsiveness depend on the intensity of the drought stress (Kim et al., 2012; Kim et al., 2008). It was concluded that SA190-induced drought proxy tolerance is mediated by enhancing H3K4me3 levels target genes in an ABA-dependent manner. Priming by beneficial microbes bears an enormous potential in agricultural engineering which suffers from the problem that expressing stress-related genes often comes with a cost in growth and yield. Given that desert plant-associated beneficial microbes, such as Enterobacter sp. SA187 can enhance salt and heat tolerance in plants (de Zelicourt et al., 2018; Shekhawat et al, 2021), desert beneficial microbes, such as SA190, might present an affordable and readily available technology to ensure food production in countries with limited water availability. On the other hand, considering the effects of global climate change, desert beneficial microbes might also help to limit yield losses in less arid regions suffering from intermittent drought stress periods.

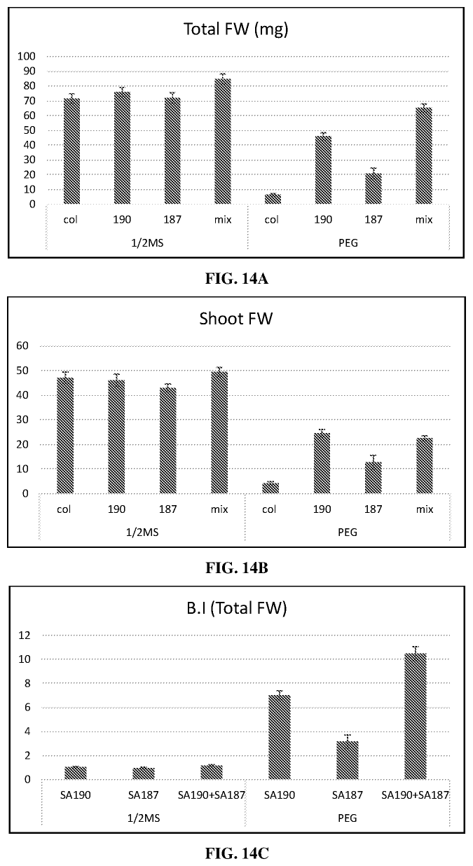

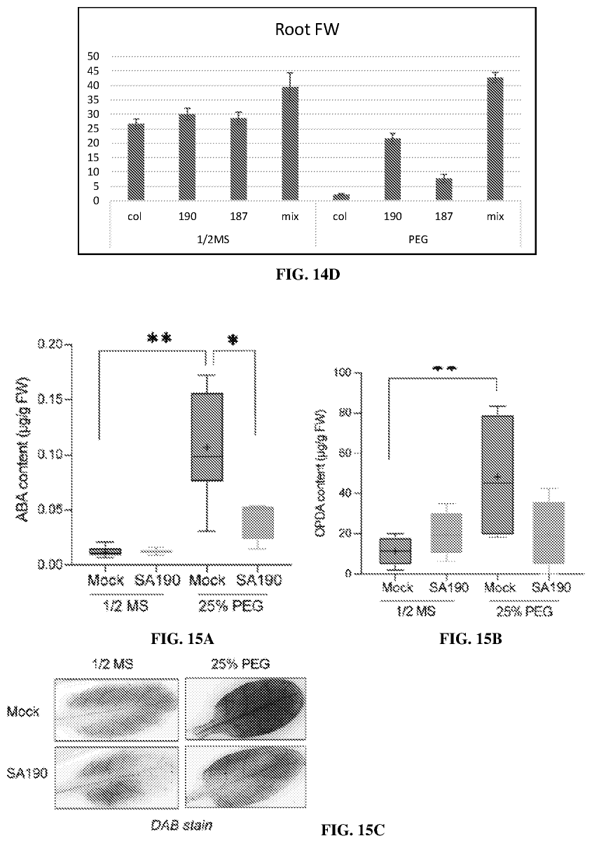

Enterobacter sp. SA187 is a microbial strain that was shown to enhance salt and heat stress tolerance of plants via the production of a bacterial metabolite that can be converted into ethylene in planta (de Zelicourt et al., 2018). SA187 showed some beneficial activity towards drought in the range of 50-60% but never achieved the 500-600% enhancement of drought stress tolerance seen with SA190. It appears that in contrast to ABA, ethylene plays no major role in the beneficial effect of SA190-induced drought stress tolerance. Therefore, we tested whether combining SA190 with SA187 could show additive beneficial activity of plant drought stress tolerance. FIG. 13 shows the experimental design used to test the effect of SA187+SA190 on Arabidopsis plants. SA190 increased the FW (Fresh weight) more than SA187 under drought stress (½ MS+25% PEG) (FIGS. 14A-14D). By contrast, SA187+SA190 mixed compared to SA187 or SA190 strains alone, showed enhanced beneficial activity on Arabidopsis under water stress conditions.

Overall, the data herein have proven that a single bacterial strain can massively enhance drought tolerance of Arabidopsis. The data shows that SA190 achieves this mainly by improving the water use efficiency and that aquaporins play a central role in this mechanism. The fact that aquaporin gene expression is not constitutive but only induced under water limiting conditions in SA190-colonized plants speaks for a priming mechanism and highlights the huge potential of applying SA190 in agricultural engineering which suffers from the problem that expressing stress related genes often comes with a cost in growth and yield. As such, beneficial microbes such as SA190 might present a break-through technology to ensure food and feed production in many countries with limited water availability but could also help to limit yield losses in regions suffering from short drought stress periods.

Limiting application of irrigation water decreased production of cucumber and alfalfa up to 17.1% and 9.4%, respectively, in comparison to well irrigation conditions. SA190 inoculation of plants caused significant increases in crop production either under normal or water stress conditions. For greenhouse grown cucumber, SA190 increased the total yield by 10% and 12% in normal and water stress conditions, respectively (FIG. 1A). Similarly, alfalfa colonized plants resulted 21.1% under normal and 14.4% under water stress conditions (FIG. 1B). In conclusion, SA190 can efficiently improve crop productivity of cucumber and alfalfa under desert agricultural production conditions.

### REFERENCES

Bokhari A, et al., (vol 9, 18154, 2019). Sci Rep-Uk 10
Bolger A M, et al., Bioinformatics 30:2114-2120
Bulgarelli D, et al., (2012) Nature 488:91-95
Busby P E, et al., (2017) PLOS Biol 15: e2001793
Chen K, et al., Journal of Integrative Plant Biology 62:25-54
Cohen A C, et al., (2015) Physiol Plant 153:79-90
Cominelli E, et al., (2013) N Biotechnol 30:355-361
Cruz de Carvalho M H (2008) Plant Signal Behav 3:156-165
Cutler S R, et al., (2010) Annu Rev Plant Biol 61:651-679
Daur I, et al., (2018) Front Microbiol 9
de Zelicourt A, et al., (2013) Mol Plant 6:242-245
de Zelicourt A, et al (2018) Plos Genet 14
Ding Y, et al., (2011) Plant J 66:735-744
Ding Y, et al., (2012a) Nature Communications 3:740
Ding Y, et al., (2012b) Plos Genet 8: e1003111
Finkel O M, et al., (2017) Curr Opin Plant Biol 38:155-163
Fujii H, et al., (2009) Nature 462:660-664
Giday H, et al., (2014) Journal of Experimental Botany 65:4361-4370
Huang D W, et al., Nature Protocols 4:44-57
Jang J Y, et al., (2004) Plant Molecular Biology 54:713-725
Johanson U, et al., (2001) Plant Physiology 126:1358-1369
Kim J-M, et al., (2012) Plant and Cell Physiology 53:847-856
Kim J-M, et al., (2008) Plant and Cell Physiology 49:1580-1588
Lafi F F, et al., (2016) Genome Announc 4
Li G, et al., (2014) Biochimica et Biophysica Acta (BBA) 1840:1574-1582
Liao Y, et al., (2013) Bioinformatics 30:923-930
Lisar S Y, et al., (2016) Drought Stress Tolerance in Plants, Vol 1
Love M I, et al., (2014) Genome Biol 15
Maurel C, et al., (2015) Physiol Rev 95:1321-1358
Moshelion M, et al., (2015) Plant Cell Environ 38:1785-1793
Nakashima K, et al., (2013) Plant Cell Rep 32:959-970
Pawlowicz I, Masajada K (2019) Gene 687:166-172
Pirasteh-Anosheh H, et al., (2016) In: pp. 24-40.
Quigley F, et al., (2002) Genome Biol 3: RESEARCH0001-RESEARCH0001
Saad M M, et al., (2020) Journal of Experimental Botany 71:3878-3901
Sade N, et al., (2015) Bio-protocol 5: e1451
Sati D, et al., (2022) Journal of Soil Science and Plant Nutrition
Seleiman M F, et al., (2021) Plants-Basel 10
Shekhawat K, et al., (2021) Embo Rep 22
Sherman B T, et al., (2022) Nucleic Acids Res 50: W216-221
Suzuki N, et al., (2014) New Phytol 203:32-43
Synek L, et al., (2021). Environ Microbiol 23:6223-6240
To T, Kim J-M (2014) Frontiers in Plant Science 4
Trapnell C, et al., (2009) Bioinformatics 25:1105-1111
van Dijk K, et al (2010) Bmc Plant Biol 10
Wituszynska W, Karpiński S (2014) Bio-protocol 4: e1041
Zhu J K (2002) Annu Rev Plant Biol 53:247-273

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Those skilled in the art will recognize or be able to ascertain using no more than routine experimentation, many equivalents to the specific embodiments of the method and compositions described herein. Such equivalents are intended to be encompassed by the following claims.

1. A composition comprising P. argentinensis strain SA190, wherein the composition is selected from the group consisting of a liquid composition further comprising cell culture medium, a freeze dried composition or a solid composition selected from the group consisting of a plant substrate, a plant seed, seedling, or a mature plant.

2. The composition of claim 1, further comprising Enterobacter sp. SA187.

3. The composition of claim 1 comprising: (a) an effective amount of SA190 to confer drought tolerance to the plant following colonization of the plant or (b) further comprising SA187 in an effective amount to confer drought tolerance to the plant following colonization of the plant.

4. The composition of claim 1 wherein the plant substrate is selected from the group consisting of soil, peat, compost, vermiculite, perlite, sand, clay and combinations thereof.

5. The composition of claim 5, further comprising one or more additional plant growth-promoting bacteria or rhizobacteria.

6. The composition of claim 1, wherein the composition is a seed coating composition containing SA190, alone or in combination with SA187.

7. The composition of claim 1, wherein the composition is a freeze-dried bacterial composition comprising a cryoprotectant.

8. The composition of claim 1 wherein the composition is a liquid composition further comprising bacterial cell culture medium.

9. A method of improving growth of a seed or plant under drought conditions comprising contacting the seed or plant with the composition of claim 1.

10. The method of claim 9, wherein the levels of one or more genes selected from the group consisting of TIP2-1, TIP2-1, TIP2-2, PIP1-1, PIP1-3, PIP1-5 and PIP2-7 is increased relative to plant geminated from an untreated seed or an untreated plant

11. The method of claim 9, comprising growing the seed or plant in a plant substrate comprising an effective amount of SA190, alone or in combination with SA187. to provide the seed or plant with tolerance to the drought conditions.

12. The method of claim 9, wherein the composition is a seed coating composition comprising gelatin, cellulose, alginate, xanthum, or a combination thereof.

13. The method of claim 9, wherein the plant substrate is selected from a group consisting of soil, peat, compost, vermiculite, perlite, sand, clay and combinations thereof.

14. The method of claim 9, further comprising contacting the plant or seed with one or more additional plant growth-promoting bacteria.

15. The method of claim 14, wherein the one or more plant growth-promoting bacteria are selected from the group consisting of Paenibacillus polymyxa strain A26, a Alcaligenes faecalis strain AF, and a combination thereof.

16. The composition of claim 1, wherein the composition is a coated plant seed comprising an effective amount of SA190 and/or SA187 to provide the seed with resistance to drought stress conditions as the seed grows.

17. (canceled)

18. The method of claim 16, wherein the plant seed is coated with SA190 and/or SA187 encapsulated with a non-toxic, biodegradable coating; and a coating adhesive.

19. The method of claim 18, wherein the coating comprises gelatin, cellulose, alginate, xanthum, or a combination thereof.

20. A method of improving water use efficiency of a plant, comprising contacting the plant or a plant seed with a composition comprising P. argentinensis strain SA190.

21. The method of claim 20, wherein the composition further comprises SA187.

22. The method of claim 20 wherein the plant is irrigated with water comprising P. argentinensis strain SA190, preferably by drip irrigation.
